SEQUENCE LISTING (4 sequences)

SEQ ID NO: 1 - DNA, 743 bp, Homo sapiens
  CDS (200)..(676)
gcagccagaaagctctggagcatcagggagactccaacttaaggcaacagcatgggtgaataagggcttcctgtggactggcaatgagaggcaaaacctggtgcttgagcactggcccctaaggcaggccttacagatctcttacactcgtggtgggaagagtttagtgtgaaactggggtggaattgggtgtccacgtatgttcccttttgccttactatatgttctgtcagtttctttcaggaaaatcttcatcttacaacttgtagggctggtgttaacttacgacttcactaactgtgactttgagaagattaaagcagcctatctcagtactatttctaaagacctgattacatatatgagtgggaccaaaagtaccgagttcaacaacaccgtctcttgtagcaatcggccacattgccttactgaaatccagagcctaaccttcaatcccaccgccggctgcgcgtcgctcgccaaagaaatgttcgccatgaaaactaaggctgccttagctatctggtgcccaggctattcggaaactcagataaatgctactcaggcaatgaagaagaggagaaaaaggaaagtcacaaccaataaatgtctggaacaagtgtcacaattacaaggattgtggcgtcgcttcaatcgacctttactgaaacaacagtaaaccatctttattatggtcatatttcacagcccaaaataaatcatctttattaagtaaaaaaaaa

SEQ ID NO: 2 - protein, 159 aa, Homo sapiens
MFPFALLYVLSVSFRKIFILQLVGLVLTYDFTNCDFEKIKAAYLSTISKDLITYMSGTKSTEFNNTVSCSNRPHCLTEIQSLTFNPTAGCASLAKEMFAMKTKAALAIWCPGYSETQINATQAMKKRRKRKVTTNKCLEQVSQLQGLWRRFNRPLLKQQ

SEQ ID NO: 3 - DNA, 1116 bp, Homo sapiens
  CDS (1)..(1116)
  sig_peptide (1)..(66)
  misc_feature (694)..(756)
atggggcggctggttctgctgtggggagctgccgtctttctgctgggaggctggatggctttggggcaaggaggagcagcagaaggagtacagattcagatcatctacttcaatttagaaaccgtgcaggtgacatggaatgccagcaaatactccaggaccaacctgactttccactacagattcaacggtgatgaggcctatgaccagtgcaccaactaccttctccaggaaggtcacacttcagggtgcctcctagacgcagagcagcgagacgacattctctatttctccatcaggaatgggacgcaccccgttttcaccgcaagtcgctggatggtttattacctgaaacccagttccccgaagcacgtgagattttcgtggcatcaggatgcagtgacggtgacgtgttctgacctgtcctacggggatctcctctatgaggttcagtaccggagccccttcgacaccgagtggcagtccaaacaggaaaatacctgcaacgtcaccatagaaggcttggatgccgagaagtgttactctttctgggtcagggtgaaggctatggaggatgtatatgggccagacacatacccaagcgactggtcagaggtgacatgctggcagagaggcgagattcgggatgcctgtgcagagacaccaacgcctcccaaaccaaagctgtccaaatttattttaatttccagcctggccatccttctgatggtgtctctcctccttctgtctttatggaaattatggagagtgaagaagtttctcattcccagcgtgccagacccgaaatccatcttccccgggctctttgagatacaccaagggaacttccaggagtggatcacagacacccagaacgtggcccacctccacaagatggcaggtgcagagcaagaaagtggccccgaggagcccctggtagtccagttggccaagactgaagccgagtctcccaggatgctggacccacagaccgaggagaaagaggcctctgggggatccctccagcttccccaccagcccctccaaggcggtgatgtggtcacaatcgggggcttcacctttgtgatgaatgaccgctcctacgtggcgttgtga

SEQ ID NO: 4 - protein, 371 aa, Homo sapiens
MGRLVLLWGAAVFLLGGWMALGQGGAAEGVQIQIIYFNLETVQVTWNASKYSRTNLTFHYRFNGDEAYDQCTNYLLQEGHTSGCLLDAEQRDDILYFSIRNGTHPVFTASRWMVYYLKPSSPKHVRFSWHQDAVTVTCSDLSYGDLLYEVQYRSPFDTEWQSKQENTCNVTIEGLDAEKCYSFWVRVKAMEDVYGPDTYPSDWSEVTCWQRGEIRDACAETPTPPKPKLSKFILISSLAILLMVSLLLLSLWKLWRVKKFLIPSVPDPKSIFPGLFEIHQGNFQEWITDTQNVAHLHKMAGAEQESGPEEPLVVQLAKTEAESPRMLDPQTEEKEASGGSLQLPHQPLQGGDVVTIGGFTFVMNDRSYVAL

(19) United States
(12) Patent Application Publication
(10) Pub. No.: US 2006/0039910 A1
(43) Pub. Date: Feb. 23, 2006

(54) Methods and compositions for treating allergic inflammation

(75) Inventors: Michael R. Comeau, Bainbridge Island, WA (US); Thibaut DeSmedt, Vilvoorde (BE); David Fitzpatrick, Seattle, WA (US)

Correspondence Address: AMGEN INC.;LAW DEPARTMENT, 1201 AMGEN COURT WEST, SEATTLE, WA, 98119, US

(73) Assignee: Amgen Inc., Thousand Oaks, CA (US)

(21) Appl. No.: 11/205,904

(22) Filed: Aug. 16, 2005

Related U.S. Application Data
(60) Provisional application No. 60/603,425, filed on Aug. 20, 2004.

(51) Int. Cl. A61K 48/00; A61K 39/395

(52) U.S. Cl. 424/145.1; 514/44

(57) ABSTRACT

The invention provides methods and compositions for treating allergic inflammation by combining cytokine antagonists capable of acting synergistically to reduce allergic inflammation in a subject. Methods of in vivo screening for therapeutically effective cytokine antagonists useful for treating allergic inflammation are also provided.

This application hereby claims benefit of U.S. provisional application Ser. No. 60/603,425, filed Aug. 20, 2004, the entire disclosure of which is relied upon and incorporated by reference.

### FIELD OF THE INVENTION

This invention relates to inflammation and in particular to treatments for allergic inflammation.

### BACKGROUND OF THE INVENTION

It has been estimated that up to twenty percent of the population of Western countries suffers from allergic diseases including asthma, allergic rhinitis, atopic dermatitis and food allergies (Kay, N Engl. J. Med. 344:30-37 (2001)). The prevalence of allergic diseases appears to be increasing in recent years, particularly in developed countries.

While the role of antigen presenting cells such as dendritic cells in establishing tolerogenic responses to allergens is well-established, these cells also appear to be involved in the pathogenesis of allergic diseases such as asthma (Lambrecht et al., Nature Rev Immunol 3, 994-1003 (2003). A typical non-pathogenic immune response to harmless allergens is a low-level immune response characterized by the production of allergen-specific IgG1 and IgG2 antibodies, and moderate proliferation and the production of interferon-γ by type 1 helper T cells (T_{H}1 cells) (Ebner et al. J Immunol 154:1932-40 (1995)). In contrast, allergic inflammation is an exaggerated, dysregulated response to otherwise harmless allergens, characterized by the production of T_{H}2-derived cytokines such as interleukin 4 (IL-4), interleukin 5 (IL-5) and interleukin 13 (IL-13) (Kay, supra). In the case of asthma, for example, these cytokines trigger induction of allergen-specific IgE antibodies, the induction of airway eosinophilia, and mucus production. Allergic responses are generally characterized by the production and infiltration of T_{H}2 cells into affected tissues, with some exceptions such as contact dermatitis (Kay, supra).

It is known that “proallergic cytokines” including IL-4, IL-5 and IL-13 promote allergic diseases by regulating both IgE synthesis and eosinophil activation. Recently, it has been reported that the epithelial cell-derived cytokine thymic stromal lymphopoietin (TSLP) acts on dendritic cells to promote allergic inflammation (Soumelis et al., Nature Immunol. 3(7) 673-680 (2002)). This study found that TSLP activates CD11c+ dendritic cells to prime naive T helper cells to produce the proallergic cytokines IL-4, IL-5, and IL-13, and induce production of the T_{H}2-attracting chemokines TARC (thymus and activation-regulating chemokine, also known as CCL17) and MDC (macrophage-derived chemokine, CCL22) (Soumelis, supra). However, the interactions between the various cytokines involved in an allergic response are not yet clearly understood. The present invention provides new treatments for allergic inflammation based on the discovery of synergistic relationships between various cytokines during allergic inflammation.

### SUMMARY OF THE INVENTION

The present invention provides methods and compositions for treating allergic inflammation by combining cytokine antagonists which act synergistically to inhibit the condition.

The present invention provides a method of reducing allergic inflammation in a subject suffering from such a condition comprising administering to the subject a therapeutically effective amount of at least one antagonist to the cytokine thymic stromal lymphopoietin (TSLP) in combination with a therapeutically effective amount of one or more antagonist to at least one additional cytokine. In one embodiment the second cytokine is selected from the proinflammatory cytokines tumor necrosis factor-alpha (TNF-α) or interleukin 1α (IL-1α). In another embodiment, the method of reducing allergic inflammation further comprises administering at least one additional antagonist to one or more one or more T_{H}2 proallergic cytokines. In one embodiment, the T_{H}2 proallergic cytokines are selected from the group consisting of IL-4, IL-5 or IL-13.

In another embodiment, the invention provides a method of reducing allergic inflammation in a subject comprising administering a therapeutically effective amount of an antagonist to TNF-α or IL-1α in combination with a therapeutically effective amount of a second antagonist or set of antagonists to one or more T_{H}2 proallergic cytokines, including, but not limited to IL-4, IL-5, or IL-13. Particular combinations of antagonists according to the present invention include but are not limited to the following combinations: a TNF-α antagonist and an IL-4 antagonist, a TNF-α antagonist and an IL-13 antagonist, an IL-1α antagonist and an IL-4 antagonist, an IL-1α antagonist and an IL-13 antagonist. In another embodiment, the invention provides a method of reducing allergic inflammation in a subject comprising administering to the subject a therapeutic amount of an antagonist to TNF-α in combination with an therapeutic amount of an antagonist to IL-1α.

The cytokine antagonists according to the present invention include those which selectively bind to either the cytokine or its receptor, thereby reducing or blocking cytokine signal transduction. Cytokine antagonists of this type include antibodies or antibody fragments which bind to the cytokine, antibodies or antibody fragments which bind to one or more subunits of the cytokine receptor, peptides or polypeptides such as soluble receptors or soluble ligands, small molecules, chemicals and peptidomimetics. Cytokine antagonists according to the present invention also include molecules which reduce or prevent expression of the cytokine or its receptor, such as, for example, antisense oligonucleotides which target mRNA, and interfering messenger RNA.

In another aspect of the invention, a pharmaceutical composition is provided comprising a combination of cytokine antagonists for treatment of allergic inflammation. In one embodiment the composition comprises a therapeutically effective amount of at least one antagonist to TSLP in combination with a therapeutically effective amount of at least one antagonist to a second cytokine, wherein the second cytokine is IL-1α or TNF-α, in a pharmaceutically acceptable carrier. In another embodiment, the composition further comprises a therapeutically effective amount of at least one antagonist to one or more T_{H}2 proallergic cytokines, wherein the cytokines are selected from IL-4, IL-5 or IL-13.

In another embodiment, a pharmaceutical composition is provided which comprises a therapeutically effective amount an antagonist to TNF-α or IL-1α in combination with a therapeutically effective amount of at least one antagonist to one or more T_{H}2 proallergic cytokines, including, but not limited to, IL-4, IL-5, or IL-13, in a pharmaceutically acceptable carrier. Particular combinations of antagonists in compositions according to the present invention include but are not limited to the following combinations: a TNF-α antagonist and an IL-4 antagonist, a TNF-α antagonist and an IL-13 antagonist, an IL-1α antagonist and an IL-4 antagonist, an IL-1α antagonist and an IL-13 antagonist. In another embodiment, the invention provides a pharmaceutical composition comprising a therapeutically effective amount of an antagonist to TNF-α in combination with a therapeutically effective amount of an antagonist to IL-1α, in a pharmaceutically acceptable carrier. In another embodiment, additional anti-inflammatory agents are administered together with the pharmaceutical compositions of the present invention. This includes non-steroidal anti-inflammatory drugs, analgesics, systemic steroids, and anti-inflammatory cytokines.

In another aspect of the invention, models and methods for screening agents in vivo for modulation of allergic inflammation are provided. In particular, a method of screening potential therapeutic antagonists to TSLP related disorders using a T_{H}2 adaptive transfer mouse model for asthma is provided.

### BRIEF DESCRIPTION OF THE FIGURES

FIG. 1A shows induction of human TSLP in human skin epithelial (EpiDermFT™) cells by cytokines added individually and in combination. FIG. 1B shows induction of human TSLP in human airway (EpiAirway™) cells by cytokines added individually and in combination.

FIG. 2 shows the production of CTACK/CCL27 in response to cytokines added individually and in combination to the in vitro model of human epithelial cells (EpiDermFT™).

FIG. 3 shows mouse BM-derived CD11c⁺ dendritic cells stained with anti-CD11c and anti-TSLPR (FIG. 3A) or anti-IL-7Rα (FIG. 3B) mAbs.

FIG. 4A shows TARC production in BM-derived DCs stimulated with TSLP. FIG. 4B shows expression of costimulatory molecules on the surface of BM-derived DCs were stimulated with 20 ng/ml of TSLP, where the dotted lines indicate isotype control, the thin line represents untreated DCs, and the thick line represents TSLP-treated DCs.

FIG. 5A shows TARC production in BM-derived DCs from wild type and IL-7Rα knock-out mice wherein the cells were stimulated in vitro with IL-7, IL-4, or TSLP. FIG. 5B shows TARC production in BM-derived DCs from WT mice when stimulated in vitro with media, TSLP, IL-7, or IL-4, in the presence of isotype control mAb or anti-TSLP mAb.

FIG. 6A shows the experimental protocol for the generation of a T_{H}2 adoptive transfer asthma model. FIG. 6B shows the total leukocyte numbers enumerated in BAL and total numbers of eosinophils calculated from BAL by flow cytometry. Results are the mean number of cells+SEM from 5 animals per group.

FIG. 7A shows TARC levels in the BAL fluid (BALF) of T_{H}2 adoptive transfer asthma model in response to intranasal exposure to OVA or OVA plus TSLP. FIG. 7B shows number of antigen specific T_{H}2 cells in BALF in response to OVA alone or OVA plus TSLP.

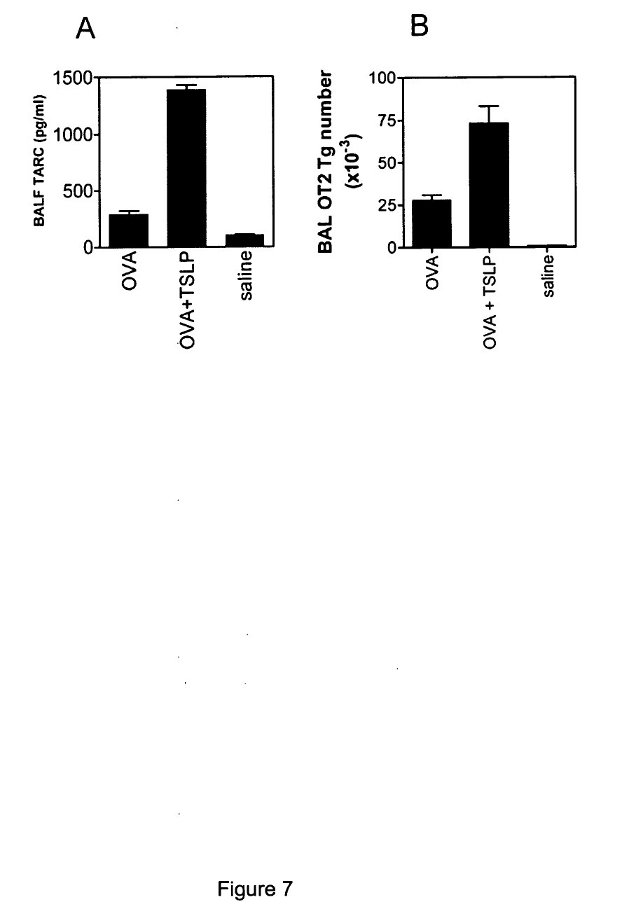

### DETAILED DESCRIPTION OF THE INVENTION

The present invention provides methods and compositions for treating inflammatory conditions.

The present invention is based on the discovery that proinflammatory cytokines such as IL-1α and tumor necrosis factor-alpha (TNF-α) induce TSLP production from the epithelial cells in various tissues, and that the production of TSLP after induction is increased synergistically by contact with T_{H}2 proallergic cytokines such as IL-4, IL-5 and IL-13 in these tissues. Additionally it has also been discovered that TSLP acts synergistically together with proinflammatory cytokines IL-1α and/or TNF-α on epithelial cells to increase production of the CTACK/CCL27, a chemokine associated with allergic inflammation, to levels much greater than those produced in response to IL-1α or TNF-α alone. Therefore, preventing or inhibiting the synergistic activity of these combinations of cytokines provides new and effective compositions and treatments for allergic inflammation. Allergic inflammation includes but is not limited to allergic rhinosinusitis, asthma, allergic conjunctivitis, and atopic dermatis.

### Combinations of Antagonists

The present invention provides a method of reducing allergic inflammation in a tissue by contacting the tissue with the various combinations of cytokine antagonists set forth below. The invention provides a method of reducing allergic inflammation a subject suffering from such a condition comprising administering to the subject a therapeutically effective amount of one or more antagonists to the cytokine thymic stromal lymphopoietin (TSLP) in combination with a therapeutically effective amount of one or more antagonists to at least one additional cytokine sufficient to obtain the desired therapeutic effect. In one embodiment the second cytokine is a proinflammatory cytokine tumor necrosis factor-alpha (TNF-α) or interleukin 1α (IL-1α). In another embodiment, the method of reducing allergic inflammation further comprises contacting the subject with a therapeutically effective amount of an additional antagonist or antagonists to one or more one or more T_{H}2 proallergic cytokines. In one embodiment, the T_{H}2 proallergic cytokines are selected from the group consisting of IL-4, IL-5 or IL-13.

In another embodiment, the invention provides a method of reducing allergic inflammation in a subject suffering from such a condition comprising administering to the subject a therapeutically effective amount of at least one antagonist to TNF-α or IL-1α in combination with a therapeutically effective amount of at least one antagonist to one or more T_{H}2 proallergic cytokines, including, but not limited to, IL-4, IL-5, or IL-13. In another embodiment, the invention provides a method of reducing allergic inflammation in a subject suffering from such a condition comprising administering to the subject a therapeutically effective amount of at least one antagonist to TNF-α in combination with a therapeutically effective amount of at least one antagonist to IL-1α. Particular combinations of antagonists according to the present invention include a TNF-α antagonist and an IL-4 antagonist, a TNF-α antagonist and an IL-13 antagonist, an IL-1α antagonist and an IL-4 antagonist, an IL-1α antagonist and an IL-13 antagonist, and a TNF-α antagonist and an IL-1α antagonist.

The present invention further provides pharmaceutical compositions comprising combinations of antagonists. In one embodiment, the pharmaceutical composition comprises a therapeutically effective amount of at least one antagonist to TSLP in combination with a therapeutically effective amount of at least one antagonist to a second cytokine, wherein the second cytokine is IL-1α or TNF-α, in a pharmaceutically acceptable carrier. In another embodiment, the composition further comprises a therapeutically effective amount of at least one additional antagonist to one or more T_{H}2 proallergic cytokines. In one embodiment, these cytokines are selected from IL-4, IL-5 or IL-13.

In another embodiment, a pharmaceutical composition is provided which comprises a therapeutically effective amount an antagonist to TNF-α or IL-1α in combination with a therapeutically effective amount of at least one antagonist to one or more T_{H}2 proallergic cytokines, including, but not limited to, IL-4, IL-5, or IL-13, in a pharmaceutically acceptable carrier. Particular combinations of antagonists in compositions according to the present invention include but are not limited to the following combinations: a TNF-α antagonist and an IL-4 antagonist, a TNF-α antagonist and an IL-13 antagonist, an IL-1α antagonist and an IL-4 antagonist, an IL-1α antagonist and an IL-13 antagonist. In another embodiment, the invention provides a pharmaceutical composition comprising a therapeutically effective amount of an antagonist to TNF-α in combination with a therapeutically effective amount of an antagonist to IL-1α, in a pharmaceutically acceptable carrier. In another embodiment, the pharmaceutical compositions may further comprise additional anti-inflammatory agents, including, for example, non-steroidal anti-inflammatory drugs, analgesics, systemic steroids, and anti-inflammatory cytokines.

In another aspect of the present invention, methods of screening potential modulating agents of allergic inflammation are also provided. These modulating agents include cytokine agonists and antagonists. As shown in Example 3 of the application, agents can be screened using murine models such as the T_{H}2 adoptive transfer mouse asthma model described below. Therefore, the present invention further provides methods of testing potential therapeutic antagonists in vivo by administering an effective amount of TSLP, with and without the potential antagonist or antagonists, to these animal models. In one embodiment, the model is an OVA-specific OT2 transgenic mouse model as described below.

As used herein the term “allergic inflammation” refers to the manifestations of immunoglobulin E (IgE)-related immunological responses. (Manual of Allergy and Immunology, Chapter 2, Alvin M. Sanico, Bruce S. Bochner, and Sarbjit S. Saini, Adelman et al, ed., Lippincott, Williams, Wilkins, Philadelphia, Pa., (2002)). Allergic inflammation as used herein is generally characterized by the infiltration into the affected tissue of type 2 helper T cells (T_{H}2 cells) (Kay, supra). Allergic inflammation includes pulmonary inflammatory diseases such as allergic rhinosinusitis, asthma, allergic conjunctivitis, in addition to inflammatory skin conditions such as atopic dermatis (Manual of Allergy and Immunology, supra). As used herein the term “TSLP-related allergic inflammation” refers to allergic inflammation conditions in which TSLP is upregulated, or has been demonstrated to be otherwise involved.

Allergic asthma is a chronic inflammatory disorder of the airways characterized by airway eosinophilia, high levels of serum IgE and mast cell activation, which contribute to airway hyperresponsiveness, epithelial damage and mucus hypersecretion (Wils-Karp, M, Ann. Rev. Immunol. 17:255-281 (1999), Manual of Allergy and Immunology, supra). Studies have demonstrated that varying degrees of chronic inflammation are present in the airways of all asthmatics, even during symptom-free periods. In susceptible individuals, this inflammation causes recurrent episodes of wheezing, breathlessness, chest tightness, and coughing. (Manual of Allergy and Immunology, supra).

Atopic dermatitis is a chronic pruritic inflammatory skin disease characterized by skin lesions, featuring an elevated serum total IgE, eosinophilia, and increased release of histamine from basophils. Persons suffering from atopic dermatitis exhibit exaggerated T_{H}2 responses and initiation of atopic dermatitis lesions is thought to be mediated by means of early skin infiltration of T_{H}2 lymphocytes releasing high levels of IL-4, IL-5 and IL-13 (Leung, J. Allergy Clin Immunol 105:860-76 (2000)).

Cytokine are low molecular weight regulatory proteins secreted in response to certain stimuli, which act on receptors on the membrane of target cells. Cytokines regulate a variety of cellular responses. Cytokines are generally described in references such as Cytokines, A. Mire-Sluis and R. Thorne, ed., Academic Press, New York, (1998). The term “proinflammatory cytokine” refers to cytokines which generally promote inflammatory processes such as IL-1 and TNF-α. As used herein the term “T_{H}2 proallergic cytokine” refers to a cytokine which is produced by T_{H}2 cells during allergic inflammation, including but not limited to IL-4, IL-5, IL-9 and IL-13. The accession numbers for the amino acid sequences of these cytokines and their specific receptors or in the alternative, the patents or patent applications in which they appear, are found in Table I below.

TABLE I

| | | | | Accession |
|---|---|---|---|---|
| | | | Database(s) | No. |
| Protein | | | (or Patent | (or SEQ |
| Name | Species | Synonyms | Application) | ID No:) |
| TSLP | Homo | Thymic stromal lymphopoietin protein | GenBank/ | AAK67940/ |
| | sapiens | | U.S. Pat. | SEQ ID |
| | | | No. 6555520 | NO: 2 |
| TSLP | Mus | Thymic stroma derived lymphopoietin; | GenBank | AAF81677 |
| | musculus | Thymic stromal derived lymphopoietin | | |
| TSLPR | Homo | Cytokine receptor-like 2 (CRL2); | US | SEQ ID |
| | sapiens | IL-XR; Thymic stromal lymphopoietin | 2002/0068323 | NO: 5 |
| | | protein receptor | | |
| TSLPR | Mus | Cytokine receptor-like factor 2; Type I | GenBank, | Q8CII9 |
| | | cytokine receptor delta 1; Cytokine | SWISSPROT | |
| | | receptor-like molecule 2 (CRLM-2); | | |
| | | Thymic stromal lymphopoietin protein | | |
| | | receptor | | |
| TNF- | Homo | Tumor necrosis factor; Tumor necrosis | GenBank, | P01375 |
| alpha | sapiens | factor ligand superfamily member 2; | SWISSPROT | |
| | | TNF-a; Cachectin | | |
| TNF- | Mus | Tumor necrosis factor; Tumor necrosis | GenBank, | P06804 |
| alpha | | factor ligand superfamily member 2; | SWISSPROT | |
| | | TNF-a; Cachectin | | |
| TNF-RI | Homo | Tumor necrosis factor receptor | GenBank, | P19438 |
| | sapiens | superfamily member 1A; p60; TNF-R1; | SWISSPROT | |
| | | p55; CD120a | | |
| | | [contains: Tumor necrosis factor binding | | |
| | | protein 1 (TBPI)] | | |
| TNF-RI | Mus | Tumor necrosis factor receptor | GenBank, | P25118 |
| | | superfamily member 1A; p60; TNF-R1; | SWISSPROT | |
| | | p55 | | |
| TNF-RII | Homo | Tumor necrosis factor receptor superfamily | GenBank, | P20333 |
| | sapiens | member 1B; Tumor necrosis factor receptor | SWISSPROT | |
| | | 2; p80; TNF-R2; p75; CD120b; Etanercept | | |
| | | [contains: Tumor necrosis factor binding | | |
| | | protein 2 (TBPII)] | | |
| TNF-RII | Mus | Tumor necrosis factor receptor | GenBank, | P25119 |
| | | superfamily member 1B; Tumor necrosis | SWISSPROT | |
| | | factor receptor 2; TNF-R2; p75 | | |
| IL-1 alpha | Homo | Interleukin-1 alpha; Hematopoietin-1 | GenBank, | P01583 |
| | sapiens | | SWISSPROT | |
| IL-1 alpha | Mus | Interleukin-1 alpha | GenBank, | P01582 |
| | | | SWISSPROT | |
| IL-1 R-1 | Homo | Interleukin-1 receptor, type I; IL-1R- | GenBank, | P14778 |
| | sapiens | alpha; P80; Antigen CD121a | SWISSPROT | |
| IL-1 R-1 | Mus | Interleukin-1 receptor, type I; P80 | GenBank, | P13504 |
| | | | SWISSPROT | |
| IL-1 R-2 | Homo | Interleukin-1 receptor, type II; IL-1R- | GenBank, | P27930 |
| | sapiens | beta; Antigen CDw121b | SWISSPROT | |
| IL-1 R-2 | Mus | Interleukin-1 receptor, type II | GenBank, | P27931 |
| | | | SWISSPROT | |
| IL-4 | Homo | Interleukin-4; B-cell stimulatory factor 1 | GenBank, | P05112 |
| | sapiens | (BSF-1); Lymphocyte stimulatory factor 1 | SWISSPROT | |
| IL-4 | Mus | Interleukin-4; B-cell stimulatory factor 1 | GenBank, | P07750 |
| | | (BSF-1); Lymphocyte stimulatory factor | SWISSPROT | |
| | | 1; IGG1 induction factor; B-cell IGG | | |
| | | differentiation factor; B-cell growth | | |
| | | factor 1 | | |
| IL-4R | Homo | Interleukin-4 receptor alpha chain (IL- | GenBank, | P24394 |
| | sapiens | 4R-alpha; CD124 antigen) | SWISSPROT | |
| | | [contains: Soluble interleukin-4 receptor | | |
| | | alpha chain (sIL4Ralpha/prot); IL-4- | | |
| | | binding protein (IL4-BP)] | | |
| IL-4R | Mus | Interleukin-4 receptor alpha chain (IL- | GenBank, | P16382 |
| | | 4R-alpha) | SWISSPROT | |
| | | [contains: Soluble interleukin-4 receptor | | |
| | | alpha chain; IL-4-binding protein (IL4- | | |
| | | BP)] | | |
| IL-5 | Homo | Interleukin-5; T-cell replacing factor | GenBank, | P05113 |
| | sapiens | (TRF); Eosinophil differentiation factor; | SWISSPROT | |
| | | B cell differentiation factor I | | |
| IL-5 | Mus | Interleukin-5; T-cell replacing factor | GenBank, | P04401 |
| | | (TRF); B-cell growth factor II (BCGF- | SWISSPROT | |
| | | II); Eosinophil differentiation factor; | | |
| | | Cytotoxic T lymphocyte inducer | | |
| IL-5R | Homo | Interleukin-5 receptor alpha chain (IL- | GenBank, | Q01344 |
| | sapiens | 5R-alpha); CD125 antigen | SWISSPROT | |
| IL-5R | Mus | Interleukin-5 receptor alpha chain (IL- | GenBank, | P21183 |
| | | 5R-alpha) | SWISSPROT | |
| IL-9 | Homo | Interleukin-9; T-cell growth factor P40; | GenBank, | P15248 |
| | sapiens | P40 cytokine | SWISSPROT | |
| IL-9 | Mus | Interleukin-9; T-cell growth factor P40; | GenBank, | P15247 |
| | | P40 cytokine | SWISSPROT | |
| IL-9R | Homo | Interleukin-9 receptor | GenBank, | Q01113 |
| | sapiens | | SWISSPROT | |
| IL-9R | Mus | Interleukin-9 receptor | GenBank, | Q01114 |
| | | | SWISSPROT | |
| IL-13 | Homo | Interleukin-13 | GenBank, | P35225 |
| | sapiens | | SWISSPROT | |
| IL-13 | Mus | Interleukin-13; T-cell activation protein | GenBank, | P20109 |
| | | P600 | SWISSPROT | |
| IL-13RA-1 | Homo | Interleukin-13 receptor alpha-1 chain | GenBank, | P78552 |
| | sapiens | (IL-13R-alpha-1); CD213a1 antigen | SWISSPROT | |
| IL-13RA-1 | Mus | Interleukin-13 receptor alpha-1 chain | GenBank, | O09030 |
| | | (IL-13R-alpha-1); Interleukin-13 binding | SWISSPROT | |
| | | protein; NR4 | | |
| IL-13RA-2 | Homo | Interleukin-13 receptor alpha-2 chain; | GenBank, | Q14627 |
| | sapiens | Interleukin-13 binding protein | SWISSPROT | |
| IL-13RA-2 | Mus | IL-13 receptor alpha 2 | GenBank | AAC33240 |

As used herein the term cytokine “antagonist” or “antagonistic agent” according to the present invention refers to an agent (i.e., molecule) which inhibits or blocks the activity of a cytokine. The term “antagonist” is used synonymously with the term “inhibitory agent”. The antagonists of the present invention act by blocking or reducing cytokine signal transduction, or by reducing or preventing expression of the cytokine or its receptor. Antagonists include agents which bind to the cytokine itself, and agents which bind one or more subunits of the cytokine receptor. For example, antagonists include antagonistic antibodies or antibody fragments which bind the cytokine itself, antagonistic antibodies or antibody fragments which bind one or more subunits of the cytokine receptor, soluble ligands which bind to the receptor, soluble receptors which bind to the cytokine, as well as small molecules, peptidomimetics, and other inhibitory agents capable of binding the cytokine or its receptor. Antagonists also include molecules which reduce or prevent expression of the cytokine, its receptor or a receptor subunit. These antagonists include antisense oligonucleotides which target mRNA, and interfering messenger RNA.

As used herein, the term “subject” refers to mammals including humans. As contemplated by the present invention the term “mammals” includes primates, domesticated animals including dogs, cats, sheep, cattle, goats, pigs, mice, rats, rabbits, guinea pigs, captive animals such as zoo animals, and wild animals. As used herein the term “tissue” refers to an organ or set of specialized cells such as skin tissue, lung tissue, and other organs.

### TSLP

Thymic stromal lymphopoetin (“TSLP”) refers to a four α-helical bundle type I cytokine most closely related to IL-7. TSLP was originally cloned from a murine thymic stromal cell line (Sims et al J. Exp. Med 192 (5), 671-680 (2000)), and was found to support early B and T cell development. Human TSLP was later cloned and found to have a 43 percent identity in amino acid sequence to the murine homolog (Quentmeier et al. Leukemia 15, 1286-1292 (2001), and U.S. Pat. No. 6,555,520, which is herein incorporated by reference). The polynucleotide and amino acid sequences of TSLP are presented in SEQ ID NO: 1 and 2 respectively of the sequence listing. TSLP was found to bind with low affinity to a receptor chain from the hematopoietin receptor family (TSLP receptor or TSLPR), which is described in U.S. patent application Ser. No. 09/895,945 (publication No: 2002/0068323). The polynucleotide and amino acid sequences of TSLPR are presented in SEQ ID NO: 3 and 4 respectively of the sequence listing. The soluble domain of the TSLPR is approximately amino acids 25 through 231 of SEQ ID NO: 4. TSLP binds with high affinity to a heterodimeric complex of TSLPR and the interleukin 7 receptor alpha IL-7Rα (Park et al., J. Exp. Med 192:5 (2000), U.S. Patent application publication number U.S. 2002/0068323). The sequence of the IL-7 receptor a is SEQ ID NO: 2 of U.S. Pat. No. 5,194,375, which is herein incorporated by reference. The sequence of the soluble domain of the IL-7 receptor a is amino acid 1 to 219 of SEQ ID NO: 2 in U.S. Pat. No. 5,194,375.

Human TSLP can be expressed in modified form, in which a furin cleavage site has been removed through modification of the amino acid sequence, as described in PCT publication No: WO 2003/032898. Modified TSLP retains activity but the full length sequence is more easily expressed in microbial or mammalian cells.

TSLP is reported to be produced in human epithelial cells in skin and airways, stromal and mast cells (Soumelis et al, supra). It has been reported that human TSLP is involved in allergic inflammation. Soumelis et al, supra reported that the TSLP heterodimer receptor complex is expressed on human CD11c+ dendritic cells (DC cells). Dendritic cell culture experiments show that TSLP binding to DC cells induces the production of T_{H}2 cell attracting chemokines TARC (thymus and activation-regulated chemokine; also known as CCL17) and MDC (macrophage-derived chemokine, also known as CCL22), and upregulates costimulatory molecules HLA-DR, CD40, CD80, CD86, and CD83 on the surface of cells. TSLP-activated DCs in cell culture induced naïve CD4⁺ (Soumelis, supra) and CD8⁺ T cell differentiation into proallergic effector cells (Gilliet et al, J. Exp. Med. 197 (8), 1059-1063 (2003)) which produce proallergic cytokines IL-4, IL-5, and IL-13 and TNF-α while down-regulating IL-10 and interferon-γ (Soumelis et al., supra, Gilliet et al., supra).

TSLP protein has been further shown to be expressed in vivo in tissue samples of inflamed tonsilar epithelial cells, and keratinocytes within the lesions of atopic dermatitis (AD) patients, and its expression is associated with Langerhans cell migration and activation, further supporting its involvement with allergic inflammation (Soumelis et al., supra). However, the relationship between TSLP and other cytokines involved in allergic inflammation have not previously been described.

As described in Example 1, proinflammatory cytokines such as IL-1α and tumor necrosis factor-alpha (TNF-α) induce TSLP production from the epithelial cells in various tissues, and production of TSLP after induction is increased synergistically by contact with T_{H}2 proallergic cytokines such as IL-4, IL-5 and IL-13 in these tissues. Additionally as described in Example 2, TSLP acts synergistically together with proinflammatory cytokines IL-1α and/or TNF-α on epithelial cells to increase production of the CTACK/CCL27, a chemokine associated with allergic inflammation, to levels much greater than those produced in response to IL-1α or TNF-α alone. Therefore, preventing or inhibiting the synergistic activity of these combinations of cytokines provides new and effective compositions and treatments for allergic inflammation. Combinations of cytokine antagonists according to the present invention which are effective include but are not limited to a TNF-α antagonist and an IL-4 antagonist, a TNF-α antagonist and an IL-13 antagonist, an IL-1α antagonist and an IL-4 antagonist, an IL-1α antagonist and an IL-13 antagonist, and a TNF-α antagonist and an IL-1α antagonist.

In another aspect of the invention, murine and human TSLP have been reported to have species-specific functions (Gilliet et al, supra, Soumelis et al, supra, Leonard, Immunol. Nature 3 (7), 605-607 (2002)). Murine TSLP was reported to support early B and T cell development while human TSLP has been reported to have no direct effects on T, B, NK, neutrophils, or mast cells, but instead to act on monocytes and CD11c+ DCs (Soumelis et al, supra). Through its activity on DCs human TSLP has been proposed to play a key early role in the initiation of allergic inflammation.

However, according to the present invention and contrary to earlier reports, it has been discovered that murine TSLP acts on murine dendritic cells to promote inflammation in the same way the human TSLP acts on human dendritic cells. Example 3 below supports this finding. Murine dendritic cells have been shown express both chains of the heterodimer receptor TSLPR/IL-7Rα. In murine dendritic cell culture, stimulation with TSLP produced TARC/CCL17 and upregulated costimulatory cell surface molecules. Furthermore, this TARC induction in cell culture was inhibited by a TSLP-specific monoclonal antibody. Intranasal administration of TSLP in addition to the antigen OVA to an OVA-specific T_{H}2 transgenic mouse model increased the number of leukocytes and eosinophils recruited into the bronchoalveolar lavage fluid (BALF) by 3 and 4 fold respectively, TARC/CCL17 levels were increased, and antigen specific T_{H}2 cells increased 3 fold over that of animals administered OVA alone. Therefore, T_{H}2 adoptive transfer animals, such as the mouse asthma model described below can be used to screen therapeutic antagonists as treatments for allergic inflammation.

### TSLP Assays

TSLP activities can be measured in an assay using BAF cells expressing human TSLPR (BAF/HTR), which require active TSLP for proliferation as described in PCT patent application WO 03/032898. The BAF/HTR bioassay utilizes a murine pro B lymphocyte cell line, which has been transfected with the human TSLP receptor (cell line obtained from Steven F. Ziegler, Benaroya Research Center, Seattle, Wash.). The BAF/HTR cells are dependent upon huTSLP for growth, and proliferate in response to active huTSLP added in test samples. Following an incubation period, cell proliferation is measured by the addition of Alamar Blue dye I (Biosource International Catalog # DAL1100, 10 uL/well). Metabolically active BAF/HRT cells take up and reduce Alamar Blue, which leads to change in the fluorescent properties of the dye. Additional assays for hTSLP activity include, for example, an assay measuring induction of T cell growth from human bone marrow by TSLP as described in U.S. Pat. No. 6,555,520. Another TSLP activity is the ability to activate STAT5 as described in the reference to Levin et al., J. Immunol. 162:677-683 (1999) and PCT application publication WO 03/032898. Additional assays include in vitro skin and airway models systems such as those described in the Example 1 and 2 below can also be used to assay the production of CTACK/CCL27 (cutaneous T-cell attracting chemokine), which is associated with inflammatory skin conditions in response to TSLP and other cytokines. In addition, murine models described in Example 3 below show an inflammatory response to TSLP and provide a model for testing potential antagonists for effectiveness in vivo.

### Particular Antagonists

The cytokine antagonists according to the present invention inhibit or block at least one activity of the relevant cytokines, or alternatively, block expression of the cytokine or its receptor. Inhibiting or blocking cytokine activity can be achieved, for example, by employing antagonists which interfere with cytokine signal transduction through its receptor. For example, antagonists which block or inhibit TSLP activity include agents which specifically bind to TSLP, agents which bind to the receptor chain (TSLPR), or agents which specifically bind to the TSLPR/IL-7Rα heterodimer, thereby blocking or reducing cytokine signal transduction. Antagonistic agents can be selected using a number of screening assays known in the art, for example, the binding assays discussed herein. Antagonists which inhibit or block an activity of the cytokine include, for example, small molecules, chemicals, peptidomimetics, antibodies, antibody fragments, peptides, polypeptides, and polynucleotides (e.g., antisense or ribozyme molecules), and the like.

### Antibodies

Antagonists include antibodies which bind to either a cytokine or its receptor and reduce or block cytokine signaling. As used herein, the term “antibody” refers to refers to intact antibodies including polyclonal antibodies (see, for example Antibodies: A Laboratory Manual, Harlow and Lane (eds), Cold Spring Harbor Press, (1988)), and monoclonal antibodies (see, for example, U.S. Pat. Nos. RE 32,011, 4,902,614, 4,543,439, and 4,411,993, and Monoclonal Antibodies: A New Dimension in Biological Analysis, Plenum Press, Kennett, McKearn and Bechtol (eds.) (1980)). As used herein, the term “antibody” also refers to a fragment of an antibody such as F(ab), F(ab′), F(ab′)₂, Fv, Fc, and single chain antibodies, or combinations of these, which are produced by recombinant DNA techniques or by enzymatic or chemical cleavage of intact antibodies. The term “antibody” also refers to bispecific or bifunctional antibodies which are an artificial hybrid antibody having two different heavy/light chain pairs and two different binding sites. Bispecific antibodies can be produced by a variety of methods including fusion of hybridomas or linking of Fab′ fragments. (See Songsivilai et al, Clin. Exp. Immunol. 79:315-321 (1990), Kostelny et al., J. Immunol. 148:1547-1553 (1992)). As used herein the term “antibody” also refers to chimeric antibodies, that is, antibodies having a human constant antibody immunoglobulin domain is coupled to one or more non-human variable antibody immunoglobulin domain, or fragments thereof (see, for example, U.S. Pat. No. 5,595,898 and U.S. Pat. No. 5,693,493). The term “antibodies” also refers to “humanized” antibodies (see, for example, U.S. Pat. No. 4,816,567 and WO 94/10332), minibodies (WO 94/09817), single chain Fv-Fc fusions (Powers et al., J. Immunol. Methods 251:123-135 (2001)), and antibodies produced by transgenic animals, in which a transgenic animal containing a proportion of the human antibody producing genes but deficient in the production of endogenous antibodies are capable of producing human antibodies (see, for example, Mendez et al., Nature Genetics 15:146-156 (1997), and U.S. Pat. No. 6,300,129). The term “antibodies” also includes multimeric antibodies, or a higher order complex of proteins such as heterdimeric antibodies. “Antibodies” also includes anti-idiotypic antibodies.

Polyclonal antibodies directed toward a cytokine or its receptor polypeptide may be produced in animals (e.g., rabbits or mice) by means of multiple subcutaneous or intraperitoneal injections of the polypeptide and an adjuvant. It may be useful to conjugate the antigen polypeptide to a carrier protein that is immunogenic in the species to be immunized, such as keyhole limpet hemocyanin, serun, albumin, bovine thyroglobulin, or soybean trypsin inhibitor. Also, aggregating agents such as alum are used to enhance the immune response. After immunization, the animals are bled and the serum is assayed for antibody titer.

Monoclonal antibodies specifically reactive with a cytokine or its receptor are produced using any method that provides for the production of antibody molecules by continuous cell lines in culture. Examples of suitable methods for preparing monoclonal antibodies include the hybridoma methods of Kohler et al., 1975, Nature 256:495-97 and the human B-cell hybridoma method (Kozbor, 1984, J. Immunol. 133:3001; Brodeur et al., Monoclonal Antibody Production Techniques and Applications 51-63 (Marcel Dekker, Inc., 1987). Also provided by the invention are hybridoma cell lines that produce monoclonal antibodies reactive with cytokines or their receptors.

Monoclonal antibodies of the invention may be modified for use as therapeutics. One embodiment is a “chimeric” antibody in which a portion of the heavy (H) and/or light (L) chain is identical with or homologous to a corresponding sequence in antibodies derived from a particular species or belonging to a particular antibody class or subclass, while the remainder of the chain(s) is/are identical with or homologous to a corresponding sequence in antibodies derived from another species or belonging to another antibody class or subclass. Also included are fragments of such antibodies, so long as they exhibit the desired biological activity. See U.S. Pat. No. 4,816,567; Morrison et al., 1985, Proc. Natl. Acad. Sci. 81:6851-55.

A monoclonal antibody may also be a “humanized” antibody. Methods for humanizing non-human antibodies are well known in the art. See U.S. Pat. Nos. 5,585,089 and 5,693,762. Generally, a humanized antibody has one or more amino acid residues introduced into it from a source that is non-human. Humanization can be performed, for example, using methods described in the art (Jones et al., 1986, Nature 321:522-25; Riechmann et al., 1998, Nature 332:323-27; Verhoeyen et al., 1988, Science 239:1534-36), by substituting at least a portion of a rodent complementarity-determining region for the corresponding regions of a human antibody.

Antibodies may also be fully human antibodies. Using transgenic animals (e.g., mice) that are capable of producing a repertoire of human antibodies in the absence of endogenous immunoglobulin production such antibodies are produced by immunization with the appropriate antigen (i.e., having at least 6 contiguous amino acids), optionally conjugated to a carrier. See, e.g., Jakobovits et al., 1993, Proc. Natl. Acad. Sci. 90:2551-55; Jakobovits et al., 1993, Nature 362:255-58; Bruggermann et al., 1993, Year in Immuno. 7:33. In one method, such transgenic animals are produced by incapacitating the endogenous loci encoding the heavy and light immunoglobulin chains therein, and inserting loci encoding human heavy and light chain proteins into the genome thereof. Partially modified animals, that is, those having less than the full complement of modifications, are then cross-bred to obtain an animal having all of the desired immune system modifications. When administered an immunogen, these transgenic animals produce antibodies with human (rather than, e.g., murine) amino acid sequences, including variable regions which are immunospecific for these antigens. See PCT App. Nos. PCT/US96/05928 and PCT/US93/06926. Additional methods are described in U.S. Pat. No. 5,545,807, PCT App. Nos. PCT/US91/245 and PCT/GB89/01207, and in European Patent Nos. 546073B1 and 546073A1. Human antibodies can also be produced by the expression of recombinant DNA in host cells or by expression in hybridoma cells as described herein. Human antibodies can also be produced from phage-display libraries (Hoogenboom et al., 1991, J. Mol. Biol. 227:381; Marks et al., 1991, J. Mol. Biol. 222:581). These processes mimic immune selection through the display of antibody repertoires on the surface of filamentous bacteriophage, and subsequent selection of phage by their binding to an antigen of choice. One such technique is described in PCT App. No. PCT/US98/17364, which describes the isolation of high affinity and functional agonistic antibodies for MPL- and msk-receptors using such an approach.

Chimeric, CDR grafted, and humanized antibodies are typically produced by recombinant methods. Nucleic acids encoding the antibodies are introduced into host cells and expressed using materials and procedures described herein. In a preferred embodiment, the antibodies are produced in mammalian host cells, such as CHO cells. Monoclonal (e.g., human) antibodies may be produced by the expression of recombinant DNA in host cells or by expression in hybridoma cells as described herein.

### Peptide/Polypeptide Antagonists

Other antagonists include specific binding agents such as polypeptides or peptides which specifically bind to the cytokine or its receptor, inhibiting or blocking cytokine signaling through its receptor, thus reducing or blocking cytokine activity. As used herein the term “polypeptide” refers to any chain of amino acids linked by peptide bonds, regardless of length or post-translational modification. The term “peptide” generally refers to a shorter chain of amino acids. Polypeptides includes natural proteins, synthetic or recombinant polypeptides and peptides as well as hybrid polypeptides. As used herein, the term “amino acid” refers to the 20 standard α-amino acids as well as naturally occurring and synthetic derivatives. A polypeptide may contain L or D amino acids or a combination thereof. As used herein the term “peptidomimetic” refers to peptide-like structures which have non-amino acid structures substituted. Peptides and polypeptides known to inhibit cytokine activity are known. Examples of peptide or polypeptide inhibitors would include peptide analogs of cytokines which compete for binding to the receptor. IL-1 polypeptide inhibitors described in U.S. Pat. No. 6,599,873, which is herein incorporated by reference, which describes glycosylated and nonglycosylated polypeptide sequences having IL-1 inhibitory activity.

The binding polypeptides and peptides of the present invention can include a sequence or partial sequence of naturally occurring proteins, randomized sequences derived from naturally occurring proteins, or entirely randomized sequences.

The polypeptide antagonists which bind to the cytokines or cytokine receptors of the present invention includes fusion proteins wherein the amino and/or carboxy termini of the peptide or polypeptide is fused to another polypeptide, a fragment thereof, or to amino acids which are not generally recognized to be part of any specific protein sequence. Examples of such fusion proteins are immunogenic polypeptides, proteins with long circulating half lives, such as immunoglobulin constant regions, marker proteins, proteins or polypeptides that facilitate purification of the desired peptide or polypeptide sequences that promote formation of multimeric proteins such as leucine zipper motifs that are useful in dimer formation/stability. Fusions of antibody fragments such as the Fc domain with a polypeptide such as a soluble domain of a cytokine receptor are well known. One example is provided in the fusion of IgF, IgA, IgM or IgE with the TNF receptor.

Binding peptides or polypeptides can be further attached to peptide linkers and carrier molecules such as an Fc region in order to dimerize the molecule and thereby enhance binding affinity. These binding agents are described in U.S. Pat. No. 6,660,843, which is hereby incorporated by reference.

### Soluble Ligands

Peptide and polypeptide antagonists include soluble ligand antagonists. As used herein the term “soluble ligand antagonist” refers to soluble peptides, polypeptides or peptidomimetics capable of binding cytokine receptor subunit, or heterodimeric receptor and blocking cytokine-receptor signal transduction. Soluble ligand antagonists include variants of the cytokine which maintain substantial homology to, but not the activity of the ligand, including truncations such an N- or C-terminal truncations, substitutions, deletions, and other alterations in the amino acid sequence, such as substituting a non-amino acid peptidomimetic for an amino acid residue. Soluble ligand antagonists, for example, may be capable of binding the cytokine receptor, but not allowing signal transduction. For the purposes of the present invention a protein is “substantially similar” to another protein if they are at least 80%, preferably at least about 90%, more preferably at least about 95% identical to each other in amino acid sequence.

### Soluble Receptors

Peptide and polypeptide antagonists further include truncated versions or fragments of the cytokine receptor, modified or otherwise, capable of specifically binding to a cytokine, and blocking or inhibiting cytokine signal transduction. These truncated versions of the cytokine receptor, for example, includes naturally occurring soluble domains, as well as variations due to proteolysis of the N- or C-termini. The soluble domain includes all or part of the extracellular domain of the receptor, alone or attached to additional peptides or modifications. Examples of soluble domains of cytokine receptors are known. One example is soluble TNFR (soluble tumor necrosis factor receptor). Soluble TNFR may be any mammalian TNRF, including murine and human, as described in U.S. Pat. No. 5,395,760, U.S. Pat. No. 5,945,397, and U.S. Pat. No. 6,201,105, all of which are herein incorporated by reference.

Soluble domains of the cytokine receptors can be provided as fusion proteins. One example of an antagonist to TNF-α is the tumor necrosis receptor-Fc fusion protein (TNFR:Fc) or a fragment thereof. TNFR:Fc is a fusion protein having all or a part of an extracellular domain of any of the TNFR polypeptides including the human p55 and p75 TNFR fused to an Fc region of an antibody, as described in U.S. Pat. No. 5,605,690, which is incorporated herein by reference.

Cytokine antagonists also include cross-linked homo or heterodimeric receptors or fragments of receptors designed to bind cytokines, also known as “cytokine traps”. Cytokine traps are fusion polypeptides capable of binding a cytokine to form a non-functional complex. A cytokine trap includes at least a cytokine binding portion of an extracellular domain of the specificity determining region of a cytokine's receptor together with a cytokine binding portion of the extracellular domain of the signal transducing component of the cytokine's receptor and a component such as an Fc which multimerizes the cytokine receptor fragments.

Specific cytokine antagonists are known. These include antagonists to TNF such as entanercept (ENBREL®), sTNF-RI, onercept, D2E7, and Remicade™, and antibodies specifically reactive with TNF-α and TNF-α receptor. Antagonists include IL-1 antagonists including IL-1ra molecules such as anakinra, Kineret®, and IL-1ra-like molecules such as IL-1Hy1 and IL-1Hy2; IL-1 “trap” molecules as described in U.S. Pat. No. 5,844,099; IL-1 antibodies; solubilized IL-1 receptor, polypeptide inhibitors to IL-1α and IL-1α receptor. Additional antagonists include antibodies to IL-4 and IL-4 receptor, antibodies to IL-5 and IL-5 receptors, and antibodies to IL-13 and IL-13 receptors.

Peptide antagonists which bind to a cytokine or its receptor may be generated by any methods known in the art including chemical synthesis, digestion of proteins, or recombinant technology. Polypeptides and peptides can be synthesized in solution or on a solid support in accordance with conventional techniques. Various automatic synthesizers are commercially available and can be used in accordance with known protocols. See, for example, Stewart and Young (supra); Tam et al., J Am Chem Soc, 105:6442, (1983); Merrifield, Science 232:341-347 (1986); Barany and Merrifield, The Peptides, Gross and Meienhofer, eds, Academic Press, New York, 1-284; Barany et al., Int J Pep Protein Res, 30:705-739 (1987); and U.S. Pat. No. 5,424,398, each incorporated herein by reference.

Solid phase peptide synthesis methods use a copoly(styrene-divinylbenzene) containing 0.1-1.0 mM amines/g polymer. These methods for peptide synthesis use butyloxycarbonyl (t-BOC) or 9-fluorenylmethyloxy-carbonyl(FMOC) protection of alpha-amino groups. Both methods involve stepwise syntheses whereby a single amino acid is added at each step starting from the C-terminus of the peptide (See, Coligan et al., Curr Prot Immunol, Wiley Interscience, 1991, Unit 9). On completion of chemical synthesis, the synthetic peptide can be deprotected to remove the t-BOC or FMOC amino acid blocking groups and cleaved from the polymer by treatment with acid at reduced temperature (e.g., liquid HF-10% anisole for about 0.25 to about 1 hours at 0° C.). After evaporation of the reagents, the peptides are extracted from the polymer with 1% acetic acid solution that is then lyophilized to yield the crude material. This can normally be purified by such techniques as gel filtration on Sephadex G-15 using 5% acetic acid as a solvent. Lyophilization of appropriate fractions of the column will yield the homogeneous peptides or peptide derivatives, which can then be characterized by such standard techniques as amino acid analysis, thin layer chromatography, high performance liquid chromatography, ultraviolet absorption spectroscopy, molar rotation, solubility, and quantitated by the solid phase Edman degradation.

Phage display and RNA-peptide screening, and other affinity screening techniques are also useful for generating peptides capable of binding cytokines or their receptors. Phage display techniques can be particularly effective in identifying peptides capable of bind ing cytokines or their receptors. Briefly, a phage library is prepared (using e.g. ml 13, fd, or lambda phage), displaying inserts from 4 to about 80 amino acid residues. The inserts may represent, for example, a completely degenerate or biased array. Phage-bearing inserts that bind to the desired antigen are selected and this process repeated through several cycles of reselection of phage that bind to the desired antigen. DNA sequencing is conducted to identify the sequences of the expressed peptides. The minimal linear portion of the sequence that binds to the desired antigen can be determined in this way. The procedure can be repeated using a biased library containing inserts containing part or all of the minimal linear portion plus one or more additional degenerate residues upstream or downstream thereof. These techniques may identify peptides with still greater binding affinity for the cytokines or their receptors. Phage display technology is described, for example, in Scott et al. Science 249: 386 (1990); Devlin et al., Science 249: 404 (1990); U.S. Pat. No. 5,223,409, issued Jun. 29, 1993; U.S. Pat. No. 5,733,731, issued Mar. 31, 1998; U.S. Pat. No. 5,498,530, issued Mar. 12, 1996; U.S. Pat. No. 5,432,018, issued Jul. 11, 1995; U.S. Pat. No. 5,338,665, issued Aug. 16, 1994; U.S. Pat. No. 5,922,545, issued Jul. 13, 1999; WO 96/40987, published Dec. 19, 1996; and WO 98/15833, published Apr. 16, 1998, each of which is incorporated herein by reference. The best binding peptides are selected for further analysis, for example, by using phage ELISA, described below, and then sequenced. Optionally, mutagenesis libraries may be created and screened to further optimize the sequence of the best binders. (Lowman, Ann Rev Biophys Biomol Struct 26:401-24 (1997)).

Other methods of generating binding peptides include additional affinity selection techniques known in the art, including “E. coli display”, “ribosome display” methods employing chemical linkage of peptides to RNA known collectively as “RNA-peptide screening.” Yeast two-hybrid screening methods also may be used to identify peptides of the invention that bind to cytokines or their receptors. In addition, chemically derived peptide libraries have been developed in which peptides are immobilized on stable, non-biological materials, such as olyethylene rods or solvent-permeable resins. Another chemically derived peptide library uses photolithography to scan peptides immobilized on glass slides. Hereinafter, these and related methods are collectively referred to as “chemical-peptide screening.” Chemical-peptide screening may be advantageous in that it allows use of D-amino acids and other analogues, as well as non-peptide elements. Both biological and chemical methods are reviewed in Wells and Lowman, Curr Opin Biotechnol 3: 355-62 (1992).

Additionally, selected peptides, peptidomimetics, and small molecules capable of binding cytokines and cytokine receptors can be further improved through the use of “rational drug design”. In one approach, the three-dimensional structure of a polypeptide of the invention, a ligand or binding partner, or of a polypeptide-binding partner complex, is determined by x-ray crystallography, by nuclear magnetic resonance, or by computer homology modeling or, most typically, by a combination of these approaches. Relevant structural information is used to design analogous molecules, to identify efficient inhibitors, such as small molecules that may bind to a polypeptide of the invention. Examples of algorithms, software, and methods for modeling substrates or binding agents based upon the three-dimensional structure of a protein are described in PCT publication WO/0107579A2, the disclosure of which is incorporated herein.

Antagonists such as peptides, polypeptides, peptidomimetics, antibodies, soluble domains, and small molecules are selected by screening for binding to the target cytokine or cytokine receptor targets, followed by non-specific and specific elution. A number of binding assays are known in the art and include non-competitive and competitive binding assays. Subsequently inhibitory parameters such as IC₅₀ (concentration at which 50% of a designated activity is inhibited) and the binding affinity as measured by K_{D} (dissociation constant) can be determined using cell-based or other assays. IC₅₀ can be determined used cell based assays, for example, employing cell cultures expressing cytokine receptors on the cell surface, as well as a cytokine-responsive signaling reporter such as a pLuc-MCS reporter vector (Stratagene cat # 219087). The inhibition of signaling when increasing quantities of antagonist is present in the cell culture along with the cytokine can be used to determine IC₅₀. AS used here the term “specifically binds” refers to a binding affinity of at least 10⁶M⁻¹, in one embodiment, 10⁷ M⁻¹ or greater. Equilibrium constant K_{D} can be determined by using BIAcore® assay systems such as BIAcore®3000 (Biacore, Inc., Piscataway, N.J.) using various concentrations of candidate inhibitors via primary amine groups using the Amine Coupling Kit (Biacore, Inc.) according to the manufacturer's suggested protocol. The therapeutic value of the inhibitory agents can then be determined by testing on various animal models such as the T_{H}2 adoptive transfer asthma model described below. Additional animal models for studying asthma, for example, is described in Lambrecht et al., Nat Rev Immunol. 3, 994-1003 (2003).

Regardless of the manner in which the peptides or polypeptides are prepared, a nucleic acid molecule encoding each peptide or polypeptide can be generated using standard recombinant DNA procedures. The nucleotide sequence of such molecules can be manipulated as appropriate without changing the amino acid sequence they encode to account for the degeneracy of the nucleic acid code as well as to account for codon preference in particular host cells. Recombinant DNA techniques also provide a convenient method for preparing polypeptide antagonists of the present invention, or fragments thereof including soluble receptor domains, for example. A polynucleotide encoding the polypeptide or fragment may be inserted into an expression vector, which can in turn be inserted into a host cell for production of the antagonists of the present invention.

A variety of expression vector/host systems may be utilized to express the peptides and polypeptide antagonists. These systems include but are not limited to microorganisms such as bacteria transformed with recombinant bacteriophage, plasmid or cosmid DNA expression vectors; yeast transformed with yeast expression vectors; insect cell systems infected with virus expression vectors (e.g., baculovirus); plant cell systems transfected with virus expression vectors (e.g., cauliflower mosaic virus, CaMV; tobacco mosaic virus, TMV) or transformed with bacterial expression vectors (e.g., Ti or pBR322 plasmid); or animal cell systems. Mammalian cells that are useful in recombinant protein productions include but are not limited to VERO cells, HeLa cells, Chinese hamster ovary (CHO) cell lines, COS cells (such as COS-7), W138, BHK, HepG2, 3T3, RIN, MDCK, A549, PC12, K562 and 293 cells.

The term “expression vector” refers to a plasmid, phage, virus or vector, for expressing a polypeptide from a polynucleotide sequence. An expression vector can comprise a transcriptional unit comprising an assembly of (1) a genetic element or elements having a regulatory role in gene expression, for example, promoters or enhancers, (2) a structural or sequence that encodes the antagonists which is transcribed into mRNA and translated into protein, and (3) appropriate transcription initiation and termination sequences. Structural units intended for use in yeast or eukaryotic expression systems preferably include a leader sequence enabling extracellular secretion of translated protein by a host cell. Alternatively, where recombinant protein is expressed without a leader or transport sequence, it may include an amino terminal methionyl residue. This residue may or may not be subsequently cleaved from the expressed recombinant protein to provide a final polypeptide product. For example, the peptides and peptibodies may be recombinantly expressed in yeast using a commercially available expression system, e.g., the Pichia Expression System (Invitrogen, San Diego, Calif.), following the manufacturer's instructions. This system also relies on the pre-pro-alpha sequence to direct secretion, but transcription of the insert is driven by the alcohol oxidase (AOX1) promoter upon induction by methanol. The secreted polypeptide is purified from the yeast growth medium using the methods used to purify the polypeptide from bacterial and mammalian cell supernatants.

Alternatively, the cDNA encoding the peptide and peptibodies may be cloned into the baculovirus expression vector pVL1393 (PharMingen, San Diego, Calif.). This vector can be used according to the manufacturer's directions (PharMingen) to infect Spodoptera frugiperda cells in sF9 protein-free media and to produce recombinant protein. The recombinant protein can be purified and concentrated from the media using a heparin-Sepharose column (Pharmacia).

Alternatively, the peptide or polypeptide may be expressed in an insect system. Insect systems for protein expression are well known to those of skill in the art. In one such system, Autographa californica nuclear polyhedrosis virus (AcNPV) can be used as a vector to express foreign genes in Spodoptera frugiperda cells or in Trichoplusia larvae. The peptide coding sequence can be cloned into a nonessential region of the virus, such as the polyhedrin gene, and placed under control of the polyhedrin promoter. Successful insertion of the peptide will render the polyhedrin gene inactive and produce recombinant virus lacking coat protein coat. The recombinant viruses can be used to infect S. frugiperda cells or Trichoplusia larvae in which the peptide is expressed (Smith et al., J Virol 46: 584 (1983); Engelhard et al., Proc Nat Acad Sci (USA) 91: 3224-7 (1994)).

In another example, the DNA sequence encoding the peptide can be amplified by PCR and cloned into an appropriate vector for example, pGEX-3X (Pharmacia). The pGEX vector is designed to produce a fusion protein comprising glutathione-S-transferase (GST), encoded by the vector, and a protein encoded by a DNA fragment inserted into the vector's cloning site. The primers for PCR can be generated to include for example, an appropriate cleavage site.

Alternatively, a DNA sequence encoding the peptide can be cloned into a plasmid containing a desired promoter and, optionally, a leader sequence (Better et al., Science 240:1041-43 (1988)). The sequence of this construct can be confirmed by automated sequencing. The plasmid can then be transformed into E. coli strain MC1061 using standard procedures employing CaCl₂ incubation and heat shock treatment of the bacteria (Sambrook et al., supra). The transformed bacteria can be grown in LB medium supplemented with carbenicillin, and production of the expressed protein can be induced by growth in a suitable medium. If present, the leader sequence can effect secretion of the peptide and be cleaved during secretion.

Mammalian host systems for the expression of recombinant peptides and polypeptides are well known to those of skill in the art. Host cell strains can be chosen for a particular ability to process the expressed protein or produce certain post-translation modifications that will be useful in providing protein activity. Such modifications of the protein include, but are not limited to, acetylation, carboxylation, glycosylation, phosphorylation, lipidation and acylation. Different host cells such as CHO, HeLa, MDCK, 293, WI38, and the like have specific cellular machinery and characteristic mechanisms for such post-translational activities and can be chosen to ensure the correct modification and processing of the introduced, foreign protein.

It is preferable that transformed cells be used for long-term, high-yield protein production. Once such cells are transformed with vectors that contain selectable markers as well as the desired expression cassette, the cells can be allowed to grow for 1-2 days in an enriched media before they are switched to selective media. The selectable marker is designed to allow growth and recovery of cells that successfully express the introduced sequences. Resistant clumps of stably transformed cells can be proliferated using tissue culture techniques appropriate to the cell line employed.

A number of selection systems can be used to recover the cells that have been transformed for recombinant protein production. Such selection systems include, but are not limited to, HSV thymidine kinase, hypoxanthine-guanine phosphoribosyltransferase and adenine phosphoribosyltransferase genes, in tk−, hgprt− or aprt− cells, respectively. Also, anti-metabolite resistance can be used as the basis of selection for dhfr which confers resistance to methotrexate; gpt which confers resistance to mycophenolic acid; neo which confers resistance to the aminoglycoside G418 and confers resistance to chlorsulfuron; and hygro which confers resistance to hygromycin. Additional selectable genes that may be useful include trpB, which allows cells to utilize indole in place of tryptophan, or hisD, which allows cells to utilize histinol in place of histidine. Markers that give a visual indication for identification of transformants include anthocyanins, β-glucuronidase and its substrate, GUS, and luciferase and its substrate, luciferin.

In some cases, the expressed polypeptides of this invention may need to be “refolded” and oxidized into a proper tertiary structure and disulfide linkages generated in order to be biologically active. Refolding can be accomplished using a number of procedures well known in the art. Such methods include, for example, exposing the solubilized polypeptide to a pH usually above 7 in the presence of a chaotropic agent. The selection of chaotrope is similar to the choices used for inclusion body solubilization, however a chaotrope is typically used at a lower concentration. Exemplary chaotropic agents are guanidine and urea. In most cases, the refolding/oxidation solution will also contain a reducing agent plus its oxidized form in a specific ratio to generate a particular redox potential which allows for disulfide shuffling to occur for the formation of cysteine bridges. Some commonly used redox couples include cysteine/cystamine, glutathione/dithiobisGSH, cupric chloride, dithiothreitol DTT/dithiane DTT, and 2-mercaptoethanol (bME)/dithio-bME. In many instances, a co-solvent may be used to increase the efficiency of the refolding. Commonly used cosolvents include glycerol, polyethylene glycol of various molecular weights, and arginine.

It is necessary to purify the peptides and polypeptide antagonists of the present invention. Protein purification techniques are well known to those of skill in the art. These techniques involve, at one level, the crude fractionation of the proteinaceous and non-proteinaceous fractions. Having separated the peptide polypeptides from other proteins, the peptide or polypeptide of interest can be further purified using chromatographic and electrophoretic techniques to achieve partial or complete purification (or purification to homogeneity). Analytical methods particularly suited to the preparation of peptibodies and peptides or the present invention are ion-exchange chromatography, exclusion chromatography; polyacrylamide gel electrophoresis; isoelectric focusing. A particularly efficient method of purifying peptides is fast protein liquid chromatography or even HPLC. The term “purified polypeptide or peptide” as used herein, is intended to refer to a composition, isolatable from other components, wherein the polypeptide or peptide is purified to any degree relative to its naturally-obtainable state. A purified peptide or polypeptide therefore also refers to a polypeptide or peptide that is free from the environment in which it may naturally occur. Generally, “purified” will refer to a peptide or polypeptide composition that has been subjected to fractionation to remove various other components, and which composition substantially retains its expressed biological activity. Where the term “substantially purified” is used, this designation will refer to a peptide or polypeptide composition in which the polypeptide or peptide forms the major component of the composition, such as constituting about 50%, about 60%, about 70%, about 80%, about 90%, about 95% or more of the proteins in the composition.

Various methods for quantifying the degree of purification of the peptide or polypeptide will be known to those of skill in the art in light of the present disclosure. These include, for example, determining the specific binding activity of an active fraction, or assessing the amount of peptide or polypeptide within a fraction by SDS/PAGE analysis. A preferred method for assessing the purity of a peptide or polypeptide fraction is to calculate the binding activity of the fraction, to compare it to the binding activity of the initial extract, and to thus calculate the degree of purification, herein assessed by a “-fold purification number.” The actual units used to represent the amount of binding activity will, of course, be dependent upon the particular assay technique chosen to follow the purification and whether or not the polypeptide or peptide exhibits a detectable binding activity.

Various techniques suitable for use in purification will be well known to those of skill in the art. These include, for example, precipitation with ammonium sulphate, PEG, antibodies (immunoprecipitation) and the like or by heat denaturation, followed by centrifugation; chromatography steps such as affinity chromatography (e.g., Protein-A-Sepharose), ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified antagonists.

### Antagonists to Polynucleotides

Cytokine antagonists according to the present invention further can include polynucleotide antagonists, including nucleic acid molecule antagonists, small molecule antagonists, peptide or polypeptide antagonists. These antagonists include antisense or sense oligonucleotides comprising a single-stranded polynucleotide sequence (either RNA or DNA) capable of binding to target mRNA (sense) or DNA (antisense) sequences. Antisense or sense oligonucleotides, according to the invention, comprise fragments of the targeted polynucleotide sequence encoding a cytokine or its receptor, transcription factors, or other polynucleotides involved in the expression of a cytokine or its receptor. Such a fragment generally comprises at least about 14 nucleotides, typically from about 14 to about 30 nucleotides. The ability to derive an antisense or a sense oligonucleotide, based upon a nucleic acid sequence encoding a given protein is described in, for example, Stein and Cohen, Cancer Res. 48:2659, 1988, and van der Krol et al. BioTechniques 6:958, 1988. Binding of antisense or sense oligonucleotides to target nucleic acid sequences results in the formation of duplexes that block or inhibit protein expression by one of several means, including enhanced degradation of the mRNA by RNAse H, inhibition of splicing, premature termination of transcription or translation, or by other means. The antisense oligonucleotides thus may be used to block expression of proteins. Antisense or sense oligonucleotides further comprise oligonucleotides having modified sugar-phosphodiester backbones (or other sugar linkages, such as those described in WO 91/06629) and wherein such sugar linkages are resistant to endogenous nucleases. Such oligonucleotides with resistant sugar linkages are stable in vivo (i.e., capable of resisting enzymatic degradation) but retain sequence specificity to be able to bind to target nucleotide sequences.

Other examples of sense or antisense oligonucleotides include those oligonucleotides which are covalently linked to organic moieties, such as those described in WO 90/10448, and other moieties that increases affinity of the oligonucleotide for a target nucleic acid sequence, such as poly-(L)-lysine. Further still, intercalating agents, such as ellipticine, and alkylating agents or metal complexes may be attached to sense or antisense oligonucleotides to modify binding specificities of the antisense or sense oligonucleotide for the target nucleotide sequence.

Antisense or sense oligonucleotides may be introduced into a cell containing the target nucleic acid by any gene transfer method, including, for example, lipofection, CaPO₄-mediated DNA transfection, electroporation, or by using gene transfer vectors such as Epstein-Barr virus or adenovirus.

Sense or antisense oligonucleotides also may be introduced into a cell containing the target nucleic acid by formation of a conjugate with a ligand-binding molecule, as described in WO 91/04753. Suitable ligand binding molecules include, but are not limited to, cell surface receptors, growth factors, other cytokines, or other ligands that bind to cell surface receptors. Preferably, conjugation of the ligand-binding molecule does not substantially interfere with the ability of the ligand-binding molecule to bind to its corresponding molecule or receptor, or block entry of the sense or antisense oligonucleotide or its conjugated version into the cell.

Alternatively, a sense or an antisense oligonucleotide may be introduced into a cell containing the target nucleic acid by formation of an oligonucleotide-lipid complex, as described in WO 90/10448. The sense or antisense oligonucleotide-lipid complex is preferably dissociated within the cell by an endogenous lipase.

Additional methods for preventing expression of targeted cytokines or cytokine receptors is RNA interference (RNAi) produced by the introduction of specific small interfering RNA (siRNA), as described, for example in Bosher et al., Nature Cell Biol 2, E31-E36 (2000).

The antagonistic nucleic acid molecules according to the present invention are capable of inhibiting or eliminating the functional activity of the cytokine in vivo or in vitro. In one embodiment, the selective antagonist will inhibit the functional activity of a cytokine by at least about 10%, in another embodiment by at least about 50%, in another embodiment by at least about 80%.

### Pharmaceutical Compositions

Pharmaceutical compositions containing combinations of therapeutic antagonists are administered to a subject to treat allergic inflammatory disorders. These disorders include, but are not limited to, allergic rhinosinusitis, asthma, allergic conjunctivitis, and atopic dermatitis.

As used herein the term “combination” refers to combined amounts of the ingredients that result in the therapeutic effect, whether administered serially or simultaneously. Such compositions comprise a therapeutically or prophylactically effective amount of each antagonist in admixture with pharmaceutically acceptable materials. Typically, the antagonist will be sufficiently purified for administration to an animal.

The pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. Suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates, other organic acids); bulking agents (such as mannitol or glycine), chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides and other carbohydrates (such as glucose, mannose, or dextrins); proteins (such as serum albumin, gelatin or immunoglobulins); coloring; flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (sucrose or sorbitol); tonicity enhancing agents (such as alkali metal halides (preferably sodium or potassium chloride, mannitol sorbitol); delivery vehicles; diluents; excipients and/or pharmaceutical adjuvants. (Remington's Pharmaceutical Sciences, 18^{th} Edition, A. R. Gennaro, ed., Mack Publishing Company, 1990).

The optimal pharmaceutical composition will be determined by one skilled in the art depending upon, for example, the intended route of administration, delivery format, and desired dosage. See for example, Remington's Pharmaceutical Sciences, supra. Such compositions may influence the physical state, stability, rate of in vivo release, and rate of in vivo clearance of the cytokine antagonist.

The primary vehicle or carrier in a pharmaceutical composition may be either aqueous or non-aqueous in nature. For example, a suitable vehicle or carrier may be water for injection, physiological saline solution or artificial cerebrospinal fluid, possibly supplemented with other materials common in compositions for parenteral administration. Neutral buffered saline or saline mixed with serum albumin are further exemplary vehicles. Other exemplary pharmaceutical compositions comprise Tris buffer of about pH 7.0-8.5, or acetate buffer of about pH 4.0-5.5, which may further include sorbitol or a suitable substitute therefore. In one embodiment of the present invention, compositions may be prepared for storage by mixing the selected composition having the desired degree of purity with optional formulation agents (Remington's Pharmaceutical Sciences, supra) in the form of a lyophilized cake or an aqueous solution. Further, the product may be formulated as a lyophilizate using appropriate excipients such as sucrose.

The pharmaceutical compositions can be selected for the condition to be treated. Treatment of skin-related allergic inflammatory conditions such as atopic dermatitis may be delivered topically, orally or delivered by injection, for example. Alternatively, the compositions intended to treat inflammatory disorders of the airway may be delivered, for example, by inhalation therapy, orally, nasally or by injection. The preparation of such pharmaceutically acceptable compositions is within the skill of the art.

The formulation components are present in concentrations that are acceptable to the site of administration. For example, buffers are used to maintain the composition at physiological pH or at slightly lower pH, typically within a pH range of from about 5 to about 8.

When parenteral administration is contemplated, the therapeutic compositions for use in this invention may be in the form of a pyrogen-free, parenterally acceptable aqueous solution comprising the cytokine antagonistic in a pharmaceutically acceptable vehicle. A particularly suitable vehicle for parenteral injection is sterile distilled water in which an antagonist is formulated as a sterile, isotonic solution, properly preserved. Yet another preparation can involve the formulation of the desired molecule with an agent, such as injectable microspheres, bio-erodible particles, polymeric compounds (polylactic acid, polyglycolic acid), beads, or liposomes, that provides for the controlled or sustained release of the product which may then be delivered via a depot injection. Hyaluronic acid may also be used, and this may have the effect of promoting sustained duration in the circulation. Other suitable means for the introduction of the desired molecule include implantable drug delivery devices.

In another aspect, pharmaceutical formulations suitable for parenteral administration may be formulated in aqueous solutions, preferably in physiologically compatible buffers such as Hanks' solution, ringer's solution, or physiologically buffered saline. Aqueous injection suspensions may contain substances that increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils, such as sesame oil, or synthetic fatty acid esters, such as ethyl oleate, triglycerides, or liposomes. Non-lipid polycationic amino polymers may also be used for delivery. Optionally, the suspension may also contain suitable stabilizers or agents to increase the solubility of the ompounds and allow for the preparation of highly concentrated solutions. In another embodiment, a pharmaceutical composition may be formulated for inhalation. For example, antagonists may be formulated as a dry powder for inhalation. Antagonists including polypeptide or nucleic acid molecule inhalation solutions may also be formulated with a propellant for aerosol delivery. In yet another embodiment, solutions may be nebulized. Pulmonary administration is further described in PCT Application No. PCT/US94/001875, which describes pulmonary delivery of chemically modified proteins.

It is also contemplated that certain formulations may be administered orally. In one embodiment of the present invention, molecules that are administered in this fashion can be formulated with or without those carriers customarily used in the compounding of solid dosage forms such as tablets and capsules. For example, a capsule may be designed to release the active portion of the formulation at the point in the gastrointestinal tract when bioavailability is maximized and pre-systemic degradation is minimized. Additional agents can be included to facilitate absorption of the antagonist molecule. Diluents, flavorings, low melting point waxes, vegetable oils, lubricants, suspending agents, tablet disintegrating agents, and binders may also be employed.

Pharmaceutical compositions for oral administration can also be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical preparations for oral use can be obtained through combining active compounds with solid excipient and processing the resultant mixture of granules (optionally, after grinding) to obtain tablets or dragee cores. Suitable auxiliaries can be added, if desired. Suitable excipients include carbohydrate or protein fillers, such as sugars, including lactose, sucrose, mannitol, and sorbitol; starch from corn, wheat, rice, potato, or other plants; cellulose, such as methyl cellulose, hydroxypropylmethylcellulose, or sodium carboxymethylcellulose; gums, including arabic and tragacanth; and proteins, such as gelatin and collagen. If desired, disintegrating or solubilizing agents may be added, such as the cross-linked polyvinyl pyrrolidone, agar, and alginic acid or a salt thereof, such as sodium alginate.

Dragee cores may be used in conjunction with suitable coatings, such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, i.e., dosage.

Pharmaceutical preparations that can be used orally also include push-fit capsules made of gelatin, as well as soft, sealed capsules made of gelatin and a coating, such as glycerol or sorbitol. Push-fit capsules can contain active ingredients mixed with fillers or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers.

Another pharmaceutical composition may involve an effective quantity of a cytokine antagonist in a mixture with non-toxic excipients that are suitable for the manufacture of tablets. By dissolving the tablets in sterile water, or other appropriate vehicle, solutions can be prepared in unit dose form. Suitable excipients include, but are not limited to, inert diluents, such as calcium carbonate, sodium carbonate or bicarbonate, lactose, or calcium phosphate; or agents such as starch, gelatin, or acacia; or lubricating agents such as magnesium stearate, stearic acid, or talc.

Additional pharmaceutical compositions will be evident to those skilled in the art, including formulations involving antagonist molecules in sustained- or controlled-delivery formulations. Techniques for formulating a variety of other sustained- or controlled-delivery means, such as liposome carriers, bio-erodible microparticles or porous beads and depot injections, are also known to those skilled in the art. See for example, PCT/US93/00829 that describes controlled release of porous polymeric microparticles for the delivery of pharmaceutical compositions. Additional examples of sustained-release preparations include semipermeable polymer matrices in the form of shaped articles, e.g. films, or microcapsules. Sustained release matrices may include polyesters, hydrogels, polylactides (U.S. Pat. No. 3,773,919, EP 58,481), copolymers of L-glutamic acid and gamma ethyl-L-glutamate (Sidman et al., Biopolymers, 22:547-556 (1983), poly (2-hydroxyethyl-methacrylate) (Langer et al., J. Biomed. Mater. Res., 15:167-277, (1981); Langer et al., Chem. Tech., 12:98-105 (1982)), ethylene vinyl acetate (Langer et al., supra) or poly-D(−)-3-hydroxybutyric acid (EP 133,988). Sustained-release compositions also include liposomes, which can be prepared by any of several methods known in the art. See e.g., Eppstein et al., PNAS (USA), 82:3688 (1985); EP 36,676; EP 88,046; EP 143,949.

The pharmaceutical composition to be used for in vivo administration typically must be sterile. This may be accomplished by filtration through sterile filtration membranes. Where the composition is lyophilized, sterilization using this method may be conducted either prior to or following lyophilization and reconstitution. The composition for parenteral administration may be stored in lyophilized form or in solution. In addition, parenteral compositions generally are placed into a container having a sterile access port, for example, an intravenous solution bag or vial having a stopper pierceable by a hypodermic injection needle.

Once the pharmaceutical composition has been formulated, it may be stored in sterile vials as a solution, suspension, gel, emulsion, solid, or a dehydrated or lyophilized powder. Such formulations may be stored either in a ready-to-use form or in a form (e.g., lyophilized) requiring reconstitution prior to administration.

In a specific embodiment, the present invention is directed to kits for producing a single-dose administration unit. The kits may each contain both a first container having a dried protein and a second container having an aqueous formulation. Also included within the scope of this invention are kits containing single and multi-chambered pre-filled syringes (e.g., liquid syringes and lyosyringes).

An effective amount of a pharmaceutical composition to be employed therapeutically will depend, for example, upon the therapeutic context and objectives. One skilled in the art will appreciate that the appropriate dosage levels for treatment will thus vary depending, in part, upon the molecule delivered, the indication for which the antagonist molecule is being used, the route of administration, and the size (body weight, body surface or organ size) and condition (the age and general health) of the patient. Accordingly, the clinician may titer the dosage and modify the route of administration to obtain the optimal therapeutic effect. A typical dosage may range from about 0.1 mg/kg to up to about 100 mg/kg or more, depending on the factors mentioned above. In other embodiments, the dosage may range from 0.1 mg/kg up to about 100 mg/kg; or 1 mg/kg up to about 100 mg/kg; or 5 mg/kg up to about 100 mg/kg. Wherein the antagonist is an antibody, a dose range in one embodiment is 0.1 to 20 mg/kg, and in another embodiment, 1-10 mg/kg. Another dose range for an antagonistic antibody is 0.75 to 7.5 mg/kg of body weight. Antibodies may be preferably injected or administered intravenously.

For any compound, the therapeutically effective dose can be estimated initially either in cell culture assays or in animal models such as mice, rats, rabbits, dogs, pigs, or monkeys. An animal model may also be used to determine the appropriate concentration range and route of administration. Such information can then be used to determine useful doses and routes for administration in humans.

The exact dosage will be determined in light of factors related to the subject requiring treatment. Dosage and administration are adjusted to provide sufficient levels of the active compound or to maintain the desired effect. Factors that may be taken into account include the severity of the inflammatory condition, whether the condition is acute or chronic, the general health of the subject, the age, weight, and gender of the subject, time and frequency of administration, drug combination(s), reaction sensitivities, and response to therapy. Long-acting pharmaceutical compositions may be administered every 3 to 4 days, every week, or biweekly depending on the half-life and clearance rate of the particular formulation.

The frequency of dosing will depend upon the pharmacokinetic parameters of the antagonist molecule in the formulation used. Typically, a composition is administered until a dosage is reached that achieves the desired effect. The composition may therefore be administered as a single dose, or as multiple doses (at the same or different concentrations/dosages) over time, or as a continuous infusion. Further refinement of the appropriate dosage is routinely made. Appropriate dosages may be ascertained through use of appropriate dose-response data. In addition, the composition may be administered prophylactically.

The route of administration of the pharmaceutical composition is in accord with known methods, e.g. orally, through injection by intravenous, intraperitoneal, intracerebral (intra-parenchymal), intracerebroventricular, intramuscular, intra-ocular, intraarterial, intraportal, intralesional routes, intramedullary, intrathecal, intraventricular, transdermal, subcutaneous, intraperitoneal, intranasal, enteral, topical, sublingual, urethral, vaginal, or rectal means, by sustained release systems or by implantation devices. Where desired, the compositions may be administered by bolus injection or continuously by infusion, or by implantation device.

Alternatively or additionally, the composition may be administered locally via implantation of a membrane, sponge, or another appropriate material on to which the desired molecule has been absorbed or encapsulated. Where an implantation device is used, the device may be implanted into any suitable tissue or organ, and delivery of the desired molecule may be via diffusion, timed-release bolus, or continuous administration.

In some cases, an antagonist of the present invention can be delivered by implanting certain cells that have been genetically engineered, using methods such as those described herein, to express and secrete the polypeptide. Such cells may be animal or human cells, and may be autologous, heterologous, or xenogeneic. Optionally, the cells may be immortalized. In order to decrease the chance of an immunological response, the cells may be encapsulated to avoid infiltration of surrounding tissues. The encapsulation materials are typically biocompatible, semi-permeable polymeric enclosures or membranes that allow the release of the protein product(s) but prevent the destruction of the cells by the patient's immune system or by other detrimental factors from the surrounding tissues.

The pharmaceutical compositions of the present invention can optionally include additional anti-inflammatory compounds useful for treating allergic inflammation including but not limited to non-steroidal anti-inflammatory drugs, analgesics, systemic steroids, or anti-inflammatory cytokines.

The invention having been described, the following examples are offered by way of illustration, and not limitation.

### EXAMPLE I

### Induction of TSLP in In Vitro Skin and Airway Models Using Combinations of Cytokines

Induction of TSLP by cytokines individually and in combination was determined using in vitro models of human skin tissue and human airway tissue. The human skin model used was the EpiDermFT™ Series 200 System (MatTek Corp., Ashland, Mass.). The EpiDermFT™ Series contains normal, human-derived epidermal keratinocytes (NHEK) and normal, human-derived dermal fibroblasts (NHFB) cultured to form a multilayered, highly differentiated model of the human dermis and epidermis.

The in vitro model of airway tissued used was the EpiAirway™ System (MatTek Corp., Ashland, Mass.), which is made from normal, human-derived tracheal/bronchial epithelial (NHBE or TBE) cells, which have been cultured to form a pseudo-stratified, highly differentiated model, which closely resembles the epithelial tissue of the human respiratory tract.

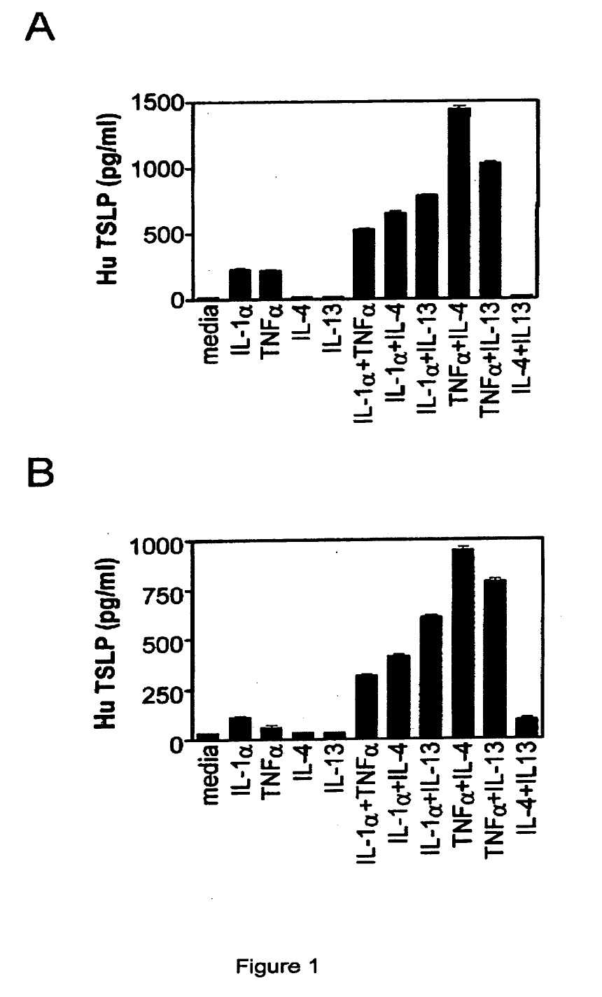

Inserts of the EpiAirway™ and EpiDermFT™ tissues respectively were each cultured with 10 ng/ml of huIL-1α, 25 ng/ml of huTNF-α, 100 ng/ml huIL-4, 100 ng/ml huIL-13 (all from R&D Systems, Minneapolis, Minn.), or the following combinations of the same human cytokines at the above concentrations: IL-1α and TNF-α, IL-1α and IL-4, IL-1α and IL-13, TNF-α and IL-4, TNF-α and IL-13, and IL-4 and IL-13. After 48 h of culturing, the supernatant was assessed for huTSLP content using a TSLP specific ELISA assay. The results are shown in FIG. 1A (skin model) and FIG. 1B (airway model).

As seen in FIG. 1, proinflammatory cytokines IL-1α and TNFα as single stimuli induced low levels of TSLP protein production in both the human airway and skin models. When used in combination, IL-1α and TNFα induced higher levels of TSLP but no significant synergy was observed compared to either cytokine alone. Neither IL-4 nor IL-13 as single stimuli alone resulted in TSLP production. However, TSLP production was dramatically increased when combinations of the proinflammatory cytokines IL-1α and TNFα and T_{H}2 proallergic cytokines IL-4 nor IL-13 were used. IL-1α or TNFα in combination with either IL-4 or IL-13 increased TSLP production 3 to 10 fold compared to any single stimuli. These results indicate that TSLP production appears to be initiated by pro-inflammatory cytokines but further amplified in the presence of T_{H}2 cytokines.

### EXAMPLE 2

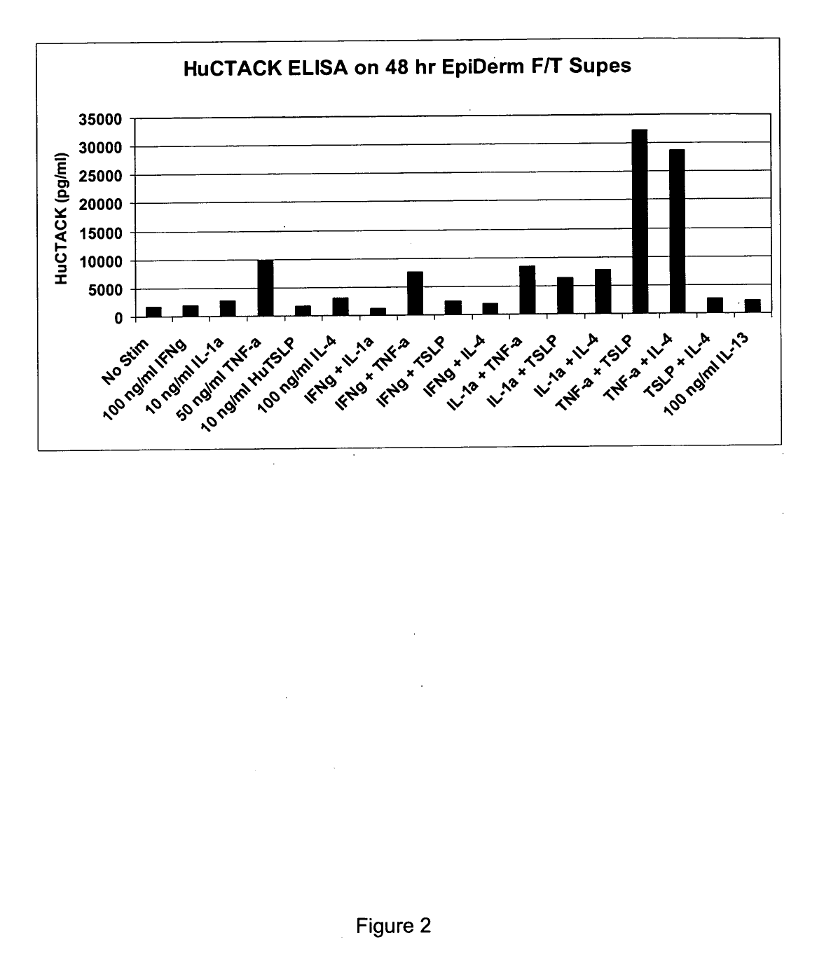

The EpiDermFT™ Series 200 was used to evaluate production of the chemokine CTACK/CCL27 (cutaneous T-cell attracting chemokine), which is the ligand for CCR10+ T cells and is associated with T-cell mediated inflammatory skin conditions including atopic dermatitis, allergic contact dermatitis, and psoriasis. Inserts of the EpiDermFT™ tissue was cultured with 100 ng/ml huIFNg, 10 ng/ml of huIL-1α, 50 ng/ml of huTNF-α, 10 ng/ml huTSLP, and 100 ng/ml huIL-4, 100 ng/ml huIL-13 (all from R&D Systems, Minneapolis, Minn.), or the following combinations of the same human cytokines at the above concentrations: IFNg and IL-1α, IFNg and TNF-α, INFg and TSLP, INFg and IL-4, IL-1α and TNF-α, IL-1α and TSLP, IL-1α and IL-4, TNF-α and TSLP, TNF-α and IL-4, and TSLP and IL-4. After 48 h of culturing, the supernatant was assessed for CTACK levels using a CTACK specific ELISA assay (R& D Systems). The results are shown in FIG. 2. It can be seen that while TNF-α alone promotes CTACK production in epithelial cells alone, TSLP acts together with TNF-α in a synergistic manner to increase the production of CTACK. Therefore, antagonizing TSLP activity in addition to TNF-α activity would effectively reduce allergic inflammation.

### EXAMPLE 3

### TSLP Function in Mice

Mouse Bone Marrow Derived Dendritic Cells Express both Chains of the Functional Receptor

Mouse bone marrow (BM) derived CD11c+ dendritic cell (DC) cultures were established as follows. Mouse BM DCs derived with FLT3L (flat-3 ligand) were obtained from female C57BL/6 WT mice 7-10 weeks of age (Jackson Laboratory, Bar Harbor, Me.) as previously described (Brawand P, J Immunol 169:6711-6719 (2002)). Cells were cultured for 10 days in McCoy's medium supplemented with 200 ng rhuFLT3L, essential and nonessential amino acids, 1 mmol/L sodium pyruvate, 2.5 mmol/L HEPES buffer (pH 7.4), vitamins, 5.5×10−5 mol/L 2-ME, 100 U/ml penicillin, 100 μg/ml streptomycin, 0.3 mg/ml L-glutamine (PSG), and 10% FBS.

To determine if the murine dendritic cells expressed one or both chains of the heterodimeric TSLP receptor, the cells were stained in FACS buffer (PBS containing 2% FBS, 1% normal rat serum, 1% normal hamster serum, 1% normal mouse serum, and 10 ug/ml 2.4G2 (a rat anti-mouse Fc receptor) mAb.). Cells were stained with anti-CD11c mAbs, and anti-TSLPR (A)(purchased from R&D Systems) or anti-IL-7Rα (B) mAbs, as shown in FIGS. 2A and 2B respectively. Flow cytometric analyses were performed on a FACSCalibur with CellQuest software (both from BD Biosciences). An electronic gate was performed on CD11c⁺ cells. Isotype controls were included (dotted lines in FIG. 2).

The results of the FACS analysis is shown in FIGS. 2A and 2B. FIG. 2A shows staining with anti-TSLPR (dotted line shows isotype controls), while FIG. 2B shows staining with anti-IL-7Rα (dotted lines show isotype controls). FIGS. 2A and 2B show strong expression of the TSLPR chain and lower levels of the IL-7Rα chain were detected on the surface of mouse dendritic cells. This indicates that mouse DCs, like human DCs, are capable of responding to TSLP.

Mouse Bone-Marrow Derived Dendritic Cells Produce TARC/CCL17 and Up Regulate Expression of Costimulatory Molecules in Response to mTSLP

It was next determined if Flt3L-derived murine bone marrow DCs could be stimulated with muTSLP to produce TARC/CCL17 as had been reported to occur for human DCs. In vitro activation of the DCs from FLT3L-supplemented cultures was accomplished by the addition of different concentrations of rmuTSLP (R&D Systems) with or without anti-TSLP mAb (R&D Systems), isotype control rat IgG2a (R&D Systems), 20 ng/ml mouse IL-7, or 20 ng/ml IL-4 (both from R&D Systems). The supernatant was collected 48 h after culture inception and assayed by ELISA for TARC content using TARC ELISA (R&D Systems).

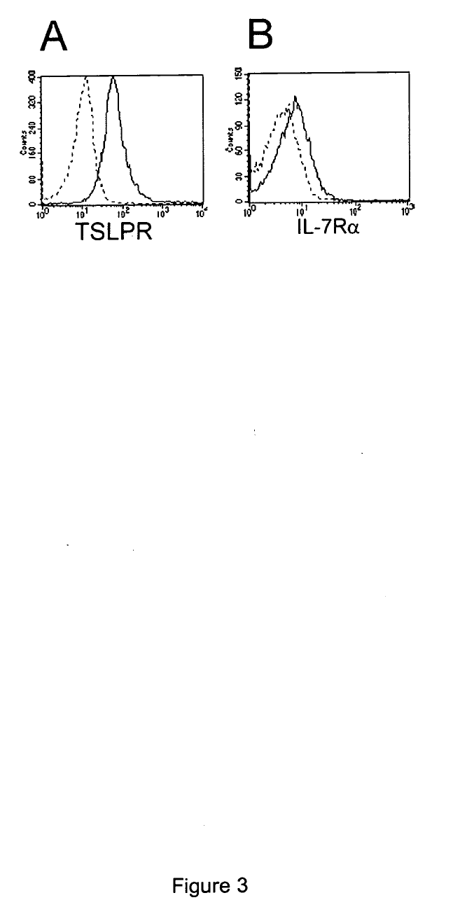

In addition, the expression of surface molecules for upregulation of co-stimulatory molecules at a 20 ng/ml murine TSLP was assessed by flow cytometry after 48 hours using monoclonal antibodies specific for MCH-ClassII(I-A^{b}), CD40, CD80, CD4, CD11c, CD86, CD90.1, CD127 (IL-7Rα, SB/199), Gr-1, and Vα2. F4/80 specific monoclonal Ab was purchased from Caltag (Burlingame, Calif.). CCR3 and TSLPR specific antibodies were purchased from R&D Systems (Minneapolis, Minn.). The results are shown in FIG. 3.

FIG. 3A shows that BM-derived DCs stimulated in vitro with graded doses of TSLP induced TARC/CCL17 production in a dose dependant manner with optimal TARC/CCL17 induction at 20 ng/ml TSLP. FIG. 3B shows that stimulating DC in vitro with 20 ng/ml of TSLP slightly up regulated expression of MHC-ClassII (I-A^{b} for mice) and CD40, while strongly increasing CD80 and CD86 surface expression compared to un-stimulated DCs. The dotted lines in FIG. 3B refer to isotype control, the thin line refers to untreated DCs; the thick line refers to TSLP-treated DCs. These results show that mouse DCs respond to TSLP in the same manner as human DCs by producing the T_{H}2 T cell attracting chemokine TARC/CCL17 and up regulating surface expression of co-stimulatory molecules. This indicates that TSLP plays a role in allergic inflammation in the mouse as well as in humans.

TSLP-Induced TARC/CCL17 Production is IL-7Rα Dependant and is Inhibited with a TSLP Specific Monoclonal Antibody.

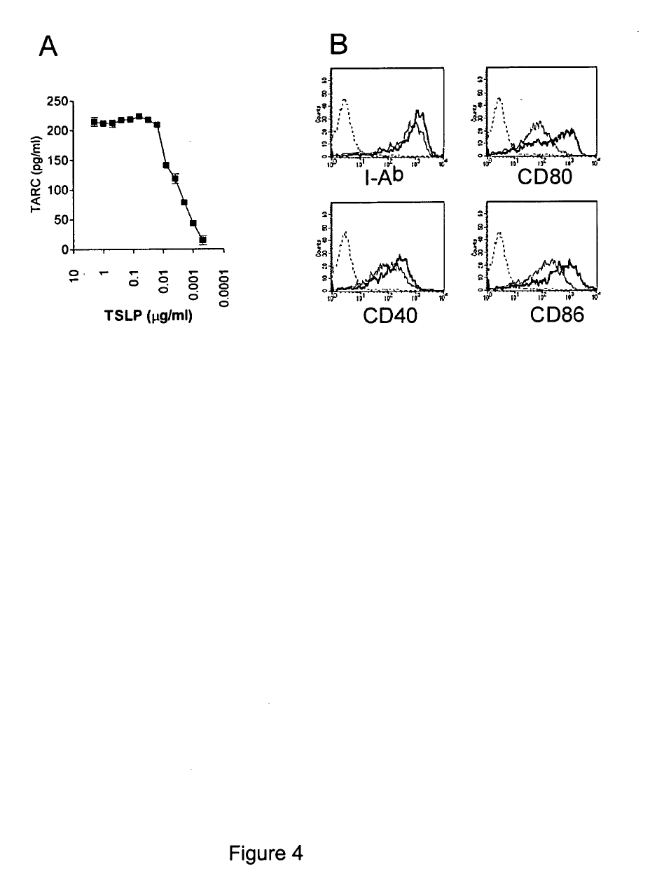

The dependence of the TSLP induced TARC/CCL17 production on the functional TSLPR heterodimer (TSLPR chain and IL-7α chain) was determined by comparing the responses of bone marrow-derived DCs from both wild type C57BL/6WT and IL-7Rα^{-/-} mice (Jackson Laboratory, Bar Harbor, Me.) to muTSLP. The results are shown in FIG. 4A. WT and IL-7Rα^{-/-} DCs both produced high levels of TARC/CCL17 in response to IL-4 as a positive control, however, only WT DCs produced TARC/CCL17 in response to both IL-7 and TSLP. IL-7 in combination with TSLP had an additive effect on WT DCs but was unable to induce TARC/CCL17 from IL-7Rα^{-/-} DCs (data not shown) further demonstrating that the presence of the IL-7Rα chain is absolutely required for TSLP induced TARC/CCL17 in mice.

To further address the specificity of the TSLP-induced TARC/CCL17 production from mouse DCs, a TSLP specific monoclonal antibody was tested for its ability to inhibit this response. Bone marrow-derived DCs were cultured 48 hrs in the presence of 20 ng/ml TSLP, IL-7, or IL-4 with or without antiTSLP mAb (denoted as α-TSLP in FIG. 4B) (R& D Systems). TARC content was assayed by ELISA in the supernatants after 48 hours. The results are shown in FIG. 4B. While the IL-7, and IL-4-induced TARC/CCL17 levels were unaffected, the TSLP-induced TARC/CCL17 production was reduced to background levels in the presence of the TSLP specific antibody as shown in FIG. 4B. An isotype matched control antibody had no affect on TARC/CCL17 production in response to any of the cytokines tested (data not shown). These data demonstrated that the TARC/CCL17 production was a TSLP specific activity.

Intranasal Administration of TSLP Protein Increases Airway Inflammation and Eosinophilia in a T_{H}2 Adoptive Transfer Asthma Model and is IL-7Rα Dependent.

The in vitro observations from Example 1 showing TSLP production from human bronchial epithelial cells following inflammatory stimuli demonstrates that TSLP plays a role in airway inflammation. In addition, TSLP specific activities on mouse DCs demonstrate that the use of mouse models is appropriate for studying TSLP-related disorders. To test this hypothesis in vivo a T_{H}2 adoptive transfer mouse model of asthma was developed. This model is an OVA-specific OT2 transgenic mouse model, as described in Cohn et al. J. Exp. Med. 190 (9), 1309-1317 (1999). The generation and adoptive transfer of OVA-specific OT2 T_{H}2 cells and measurement of airway inflammation was performed as follows. Female OT2 transgenic (Tg) mice specific for chicken OVA peptide 323-339 (OT2p) in the context of I-A^{b} were crossed with congenic B6.PL-Thy1a/Cy mice (Thy1.1 mice were obtained from the Jackson Laboratory (Bar Harbor, Me.)) to produce OT2 CD90.1 transgenic mice.

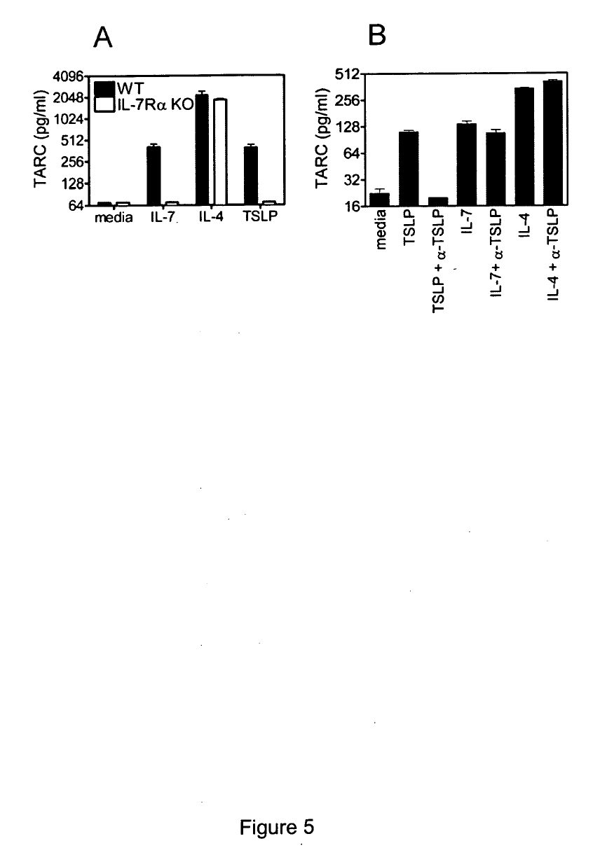

Lymph node and spleen cells from OT2 CD90.1 mice were pooled and cultured in T_{H}2 polarizing conditions for 4 days (OVA peptide 5 ug/ml, IL-2 1 ng/ml, IL-4 20 ng/ml, anti-IFN-α 10 ug/ml, anti-IL12 p70 1 ug/ml). At the end of the culture, CD4⁺ cells were isolated by negative selection (StemSep CD4⁺ T cell enrichment kit, StemCell Technologies, Vancouver, BC) and 1×10⁶ cells were injected intravenously in naïve C57BL/6 WT and IL-7Rα^{-/-} mice (Jackson Laboratory, Bar Harbor, Me.). Starting two days after transfer, mice were challenged by intranasal instillation of 100 ug OVA (chicken egg albumin, EMD Biosciences, San Diego, Calif.) with or without 200 ng mTSLP (R&D Systems) for 3 consecutive days. Two days after the last antigen challenge, mice were euthanized by avertin overdose followed by exsanguination. The experimental design is outlined in FIG. 5A. The contents of the BAL (bronchoalveolar lavage) were determined with 2×0.5 ml Ca²⁺- and Mg²⁺-free HBSS supplemented with EDTA. BALs were centrifuged and cells were resuspended in FACS buffer. Differential cell counts were performed by flow cytometric analysis. Total leukocyte numbers were enumerated in BAL and total numbers of eosinophils were calculated from BAL by flow cytometry.

Eosinophils were identified as CCR3⁺ CD11b⁺ F4/80⁻ and Gr-1^{int} cells and OT2 CD90.1 TCR Tg cells were identified as CD4⁺ Vα2⁺ and CD90.1⁺ cells. BAL fluid (BALF) was assayed for TARC content by ELISA (R&D Systems). Results are the mean number of cells+SEM from 5 animals per group.

The results were as follows. Intranasal administration of OVA in the presence of purified muTSLP protein into mice that had received adoptively transferred T_{H}2 T cells increased the total number of leukocytes recruited into the lungs 3 fold compared to administration of OVA alone (FIG. 5B). Analysis of individual cell populations indicate eosinophils recruited into the lung were increased 4 fold in the OVA+TSLP group compared to OVA alone (FIG. 5B). When IL-7Rα⁻ recipient mice were used in this system there was no TSLP-induced increase in either total cell or eosinophil numbers into the lungs of challenged mice (FIG. 5B). This demonstrated that this system of in vivo TSLP-induced airway cell recruitment is dependent on the IL-7Rα chain.

Intranasal Administration of TSLP Protein Increases TARC/CCL17 Levels and the Number of Antigen Specific T_{H}2 Cells in BALF.

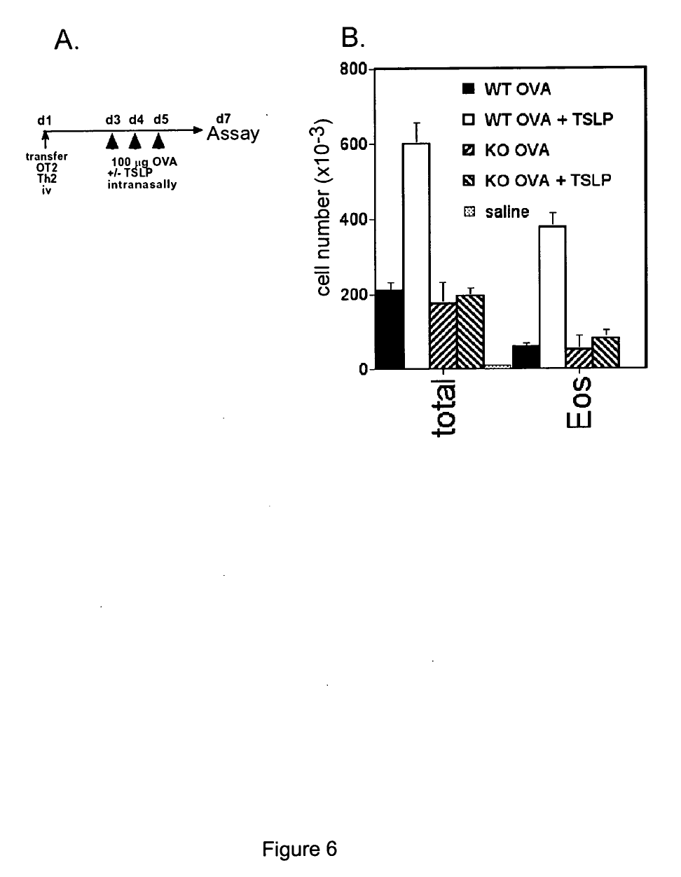

It has been demonstrated that TSLP induced TARC/CCL17 production from primary dendritic cell cultures in vitro for both human (Reche et al. J. Immunol. 167:336-343 (2001) and mouse (examples above). To determine if TSLP administration in vivo leads to increased levels of TARC/CCL17, the bronchoalveolar lavage fluid (BALF) collected from the T_{H}2 adoptive transfer model described above was analyzed. Two days after transfer, recipients were exposed to intranasal instillation of 100 ug OVA with or without 200 ng TSLP for three consecutive days. TARC/CCL17 levels was assessed by ELISA in BALF 48 h after last challenge OVA+TSLP administration led to statistically significant increased levels of TARC/CCL17 compared with animals administered OVA alone. This can be seen in FIG. 6A. Total numbers of OVA-specific OT2 Tg were calculated from BAL by flow cytometry. Results are the mean number of cells+SEM from 5 animals per group. FIG. 6B shows that the number of antigen-specific T_{H}2 cells recruited to the airways was increased 3-fold when TSLP was co-administered with OVA compared to OVA alone (FIG. 6B). This demonstrated that the TSLP acts to increase the levels of the chemokine TARC/CCL17, an indication of allergic inflammation, in vivo in the mouse T_{H}2 adoptive transfer asthma model.

The present invention is not to be limited in scope by the specific embodiments described herein, which are intended as single illustrations of individual aspects of the invention, and functionally equivalent methods and components are within the scope of the invention. Indeed, various modifications of the invention, in addition to those shown and described herein will become apparent to those skilled in the art from the foregoing description and accompanying drawings. Such modifications are intended to fall within the scope of the appended claims.

What is claimed is:

1. A method of reducing allergic inflammation in a subject suffering from such a condition comprising administering to the subject a therapeutically effective amount of at least one thymic stromal lymphpoietin (TSLP) antagonist in combination with a therapeutically effective amount of one or more antagonists to at least one additional second cytokine, wherein the second cytokine is selected from the group consisting of IL-1α and TNF-α.

2. The method of claim 1, further comprising administering one or more additional antagonists to one or more T_{H}2 proallergic cytokines.

3. The method of claim 2 wherein the T_{H}2 proallergic cytokine is selected from the group consisting of IL-4, IL-5, and IL-13.

4. The method of claim 1, wherein the cytokine antagonists are each independently selected from the group consisting of antibodies, antibody fragments, peptides, polypeptides, oligonucleotides, small molecules, chemicals and peptidomimetics.

5. The method of claim 1, wherein the antagonist specifically binds to TSLP.

6. The method of claim 5, wherein the antagonist is an antibody or an antibody fragment.

7. The method of claim 1, wherein the antagonist specifically binds to the TSLP receptor.

8. The method of claim 7, wherein the antagonist is an antibody or antibody fragment.

9. The method of claim 2, wherein the cytokine antagonists are each independently selected from the group consisting of antibodies, antibody fragments, peptides, polypeptides, oligonucleotides, small molecules, chemicals and peptidomimetics.

10. A method of reducing allergic inflammation in a subject suffering from such a condition comprising administering to the subject a therapeutically effective amount of an antagonist to TNF-α or IL-1α in combination with a therapeutically effective antagonist to one or more T_{H}2 proallergic cytokines, wherein the proallergic cytokines are selected from the group consisting of IL-4, IL-5 and IL-13.

11. The method of claim 10, wherein the combination of antagonists is selected from the group consisting of a TNF-α antagonist and an IL-4 antagonist, a TNF-α antagonist and an IL-13 antagonist, an IL-1α antagonist and an IL-4 antagonist, and an IL-1α antagonist and an IL-13 antagonist.

12. The method of claim 10 wherein the cytokine antagonists are each independently selected from the group consisting of antibodies, antibody fragments, peptides, polypeptides, oligonucleotides, small molecules, chemicals and peptidomimetics.

13. A method of reducing allergic inflammation in a subject suffering from such a condition comprising administering to the subject a therapeutically effective amount of one or more TNF-α antagonists in combination with a therapeutically effective amount of one or more IL-1α antagonists.

14. The method of claim 13, wherein the cytokine antagonists are each independently selected from the group consisting of antibodies, antibody fragments, peptides, polypeptides polynucleotides, small molecules, chemicals and peptidomimetics.

15. The methods of any one of claims 1, 10 or 13, wherein the allergic inflammation is selected from the group consisting of allergic asthma, allergic rhinosinusitis, allergic conjunctivitis, and atopic dermatitis.

16. A pharmaceutical composition for treating allergic inflammation comprising a therapeutically effective amount of one or more thymic stromal lymphopoietin (TSLP) antagonists in combination with a therapeutically effective amount of one or more antagonists to a second cytokine, wherein the second cytokine is selected from the group consisting of IL-1α or TNF-α, in a pharmaceutically acceptable carrier.

17. The composition of claim 16 further comprising a therapeutically effective amount of an additional antagonist to one or more T_{H}2 proallergic cytokines.

18. The composition of claim 17, wherein the T_{H}2 proallergic cytokine is selected from the group consisting of IL-4, IL-5 or IL-13.

19. The composition of claim 16, wherein the cytokine antagonists are each independently selected from the group consisting of antibodies, antibody fragments, peptides, polypeptides, oligonucleotides, small molecules, chemicals and peptidomimetics.

20. A pharmaceutical composition for treating allergic inflammation comprising a therapeutically effective amount of at least one antagonist to TNF-α or IL-1α in combination with a therapeutically effective amount of at least one antagonist to one or more T_{H}2 proallergic cytokines, wherein the proallergic cytokines are selected from the group consisting of IL-4, IL-5 and IL-13, in a pharmaceutically acceptable carrier.

21. The composition of claim 20, wherein the combination of antagonists is selected from the group consisting of a TNF-α antagonist and an IL-4 antagonist, a TNF-α antagonist and an IL-13 antagonist, an IL-1α antagonist and an IL-4 antagonist, and an IL-1α antagonist and an IL-13 antagonist.

22. The composition of claim 20, wherein the cytokine antagonists are each independently selected from the group consisting of antibodies, peptides, polypeptides, oligonucleotides, small molecules, chemicals and peptidomimetics.

23. A pharmaceutical composition for treating allergic inflammation comprising a therapeutically effective amount of one or more antagonists to TNF-α in combination with one or more antagonists to IL-1α, in a pharmaceutically acceptable carrier.

24. The composition of claim 23, wherein the cytokine antagonists are each independently selected from the group consisting of antibodies, peptides, polypeptides, oligonucleotides, small molecules, chemicals and peptidomimetics.

25. An in vivo method of screening agents for modulation of allergic inflammation comprising administering an appropriate dosage of thymic stromal lymphopoietin, with and without the agent, to a T_{H}2 adoptive transfer mouse.

26. The method of claim 24, wherein the mouse is an OVA-specific OT2 transgenic mouse.
